SEQUENCE LISTING (14 sequences)

SEQ ID NO: 1 - DNA, 25 bp, Artificial
  Primer
cgggatccatgagttctttcgaggg

SEQ ID NO: 2 - DNA, 31 bp, Artificial
  Primer
ataagaatgcggccgcttaggtatctaagag

SEQ ID NO: 3 - DNA, 28 bp, Artificial
  Primer
ggggtaccgctatgagttctttcgaggg

SEQ ID NO: 4 - DNA, 35 bp, Artificial
  Primer
ataagaatgcggccgccaggtatctaagagtgagc

SEQ ID NO: 5 - DNA, 31 bp, Artificial
  Primer
caaagtgtatatttgaatactacgttctgtg

SEQ ID NO: 6 - DNA, 31 bp, Artificial
  Primer
cacagaacgtagtattcaaatatacactttg

SEQ ID NO: 7 - DNA, 35 bp, Artificial
  Synthetically generated oligonucleotide
tttttttttttttttactgagtcctacagaaggat

SEQ ID NO: 8 - DNA, 21 bp, Artificial
  Synthetically generated oligonucleotide
gatccttctgtaggactcagt

SEQ ID NO: 9 - DNA, 36 bp, Artificial
  Synthetically generated oligonucleotide
actgagtcctacagaaggatcttttttttttttttt

SEQ ID NO: 10 - DNA, 40 bp, Artificial
  Synthetically generated oligonucleotide
gattactacggtagtagctacgtagctctaccgtagtaat

SEQ ID NO: 11 - DNA, 46 bp, Artificial
  Synthetically generated oligonucleotide
cgactgcgtctagacagctcacccggccgggtgagctgtctagacg

SEQ ID NO: 12 - DNA, 24 bp, Artificial
  Synthetically generated oligonucleotide
cggccgtacagtctgatcgctcat

SEQ ID NO: 13 - DNA, 25 bp, Artificial
  Synthetically generated oligonucleotide
gatgagcgatcagactgtacggccg

SEQ ID NO: 14 - protein, 692 aa, Homo sapiens <s300> Moshous, D. et al. Artemis, a novel DNA double-strand break repair/V(D)J recombination protein, is mutated in human severe combined immune deficiency Cell 105 177-186 2001-04-20
MSSFEGQMAEYPTISIDRFDRENLRARAYFLSHCHKDHMKGLRAPTLKRRLECSLKVYLYCSPVTKELLLTSPKYRFWKKRIISIEIETPTQISLVDEASGEKEEIVVTLLPAGHCPGSVMFLFQGNNGTVLYTGDFRLAQGEAARMELLHSGGRVKDIQSVYLDTTFCDPRFYQIPSREECLSGVLELVRSWITRSPYHVVWLNCKAAYGYEYLFTNLSEELGVQVHVNKLDMFRNMPEILHHLTTDRNTQIHACRHPKAEEYFQWSKLPCGITSRNRIPLHIISIKPSTMWFGERSRKTNVIVRTGESSYRACFSFHSSYSEIKDFLSYLCPVNAYPNVIPVGTTMDKVVEILKPLCRSSQSTEPKYKPLGKLKRARTVHRDSEEEDDYLFDDPLPIPLRHKVPYPETFHPEVFSMTAVSEKQPEKLRQTPGCCRAECMQSSRFTNFVDCEESNSESEEEVGIPASLQGDLGSVLHLQKADGDVPQWEVFFKRNDEITDESLENFPSSTVAGGSQSPKLFSDSDGESTHISSQNSSQSTHITEQGSQGWDSQSDTVLLSSQERNSGDITSLDKADYRPTIKENIPASLMEQNVICPKDTYSDLKSRDKDVTIVPSTGEPTTLSSETHIPEEKSLLNLSTNADSQSSSDFEVPSTPEAELPKREHLQYLYEKLATGESIAVKKRKCSLLDT

(19) United States
(12) Patent Application Publication
(10) Pub. No.: US 2009/0017010 A1
(43) Pub. Date: Jan. 15, 2009

(54) NOVEL ARTEMIS/DNA-DEPENDENT PROTEIN KINASE COMPLEX AND METHODS OF USE THEREOF

(75) Inventors: Michael R. Lieber, Los Angeles, CA (US); Yunmei Ma, Los Angeles, CA (US); Ulrich Pannicke, Ulm (DE); Klaus Schwarz, Ulm (DE)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/138,330

(22) Filed: Jun. 12, 2008

Related U.S. Application Data
(62) Division of application No. 10/359,360, filed on Feb. 5, 2003, now abandoned.
(60) Provisional application No. 60/355,452, filed on Feb. 6, 2002.
(60) Provisional application No. 60/360,659, filed on Feb. 28, 2002.

(51) Int. Cl. A61K 39/395; C12N 9/16; C07H 19/00; A61P 35/00; C12Q 1/68; C12P 21/00

(52) U.S. Cl. 424/130.1; 435/196; 536/55.3; 435/6; 435/69.1

(57) ABSTRACT

In the present invention, it is disclosed that Artemis forms a complex with the 469 kDa DNA-dependent protein kinase (DNA-PK_{cs}) in vitro and in vivo in the absence of DNA. The purified Artemis protein alone possesses single-strand specific 5′ to 3′ exonuclease activity. Upon complex formation, DNA-PK_{cs} phosphorylates Artemis, and Artemis acquires endonucleolytic activity with respect to single-stranded nucleotides, including 5′ and 3′ overhangs, as well as hairpins. Further, the Artemis:DNA-PKcs complex can open hairpins generated by the RAG complex from a 12/23-substrate pair. Thus, DNA-PK_{cs} regulates Artemis by both phosphorylation and complex formation to permit enzymatic activities that are critical for the hairpin opening step of V(D)J recombination and for all of the 5′ and 3′ overhang processing in nonhomologous DNA end joining.

### CROSS-REFERENCE TO RELATED APPLICATIONS

This application claims priority to U.S. Provisional Application Ser. No. 60/355,452, filed Feb. 6, 2002, and to U.S. Provisional Application Ser. No. 60/360,659, filed Feb. 28, 2002.

### FUNDING

This work was supported in part by NIH grant No. 5R01GM43236 to M.R.L.

### FIELD OF THE INVENTION

This invention relates to the study of nucleases. In particular, one aspect of this invention relates to the discovery of the exonuclease activity of the protein Artemis and methods of utilizing this exonuclease activity. Another aspect of this invention relates to the discovery that a complex of Artemis and the catalytic subunit of DNA-dependent protein kinase shows 5′ and 3′ overhang endonuclease and hairpin endonuclease activity. This invention further relates to the development of new medical and diagnostic applications based on Artemis and the Artemis/DNA-dependent protein kinase complex.

### BACKGROUND OF THE INVENTION

Throughout this application, various publications are referenced by author and date. Full citations for these publications may be found listed at the end of the specification immediately preceding the claims. The disclosures of these publications are hereby incorporated by reference in their entireties into this application in order to more fully describe the state of the art as known to those skilled therein as of the date of this invention described and claimed herein.

The vertebrate immune system employs a wide variety of antigen-specific receptors—the immunoglobulins and T-cell receptors—to recognize and neutralize foreign invaders. The receptor diversity necessary to recognize an almost limitless universe of potential pathogens is created by a site-specific DNA rearrangement process termed V(D)J recombination. This unique process assembles immunoglobulin and T-cell receptor variable domain exons from separate V (variable), D (diverse), and J (joint) gene segments in bone marrow pre-B cells and thymic pre-T cells, respectively (Fugmann et al., 2000; Grawunder et al., 1998; Lewis, 1994). V(D)J recombination is critical for the development of the immune system, and human patients deficient for this process manifest severe combined immune deficiency (SCID) (Schwarz et al., 1991; Schwarz et al., 1996; Vanasse et al., 1999; Villa et al., 2001).

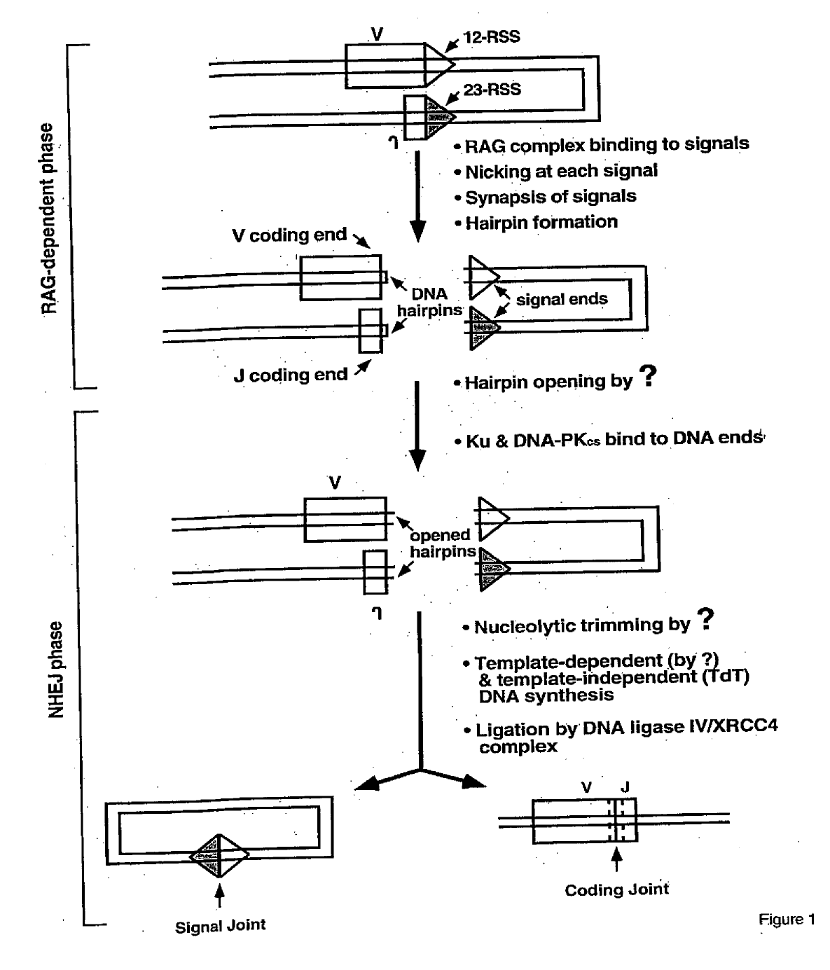

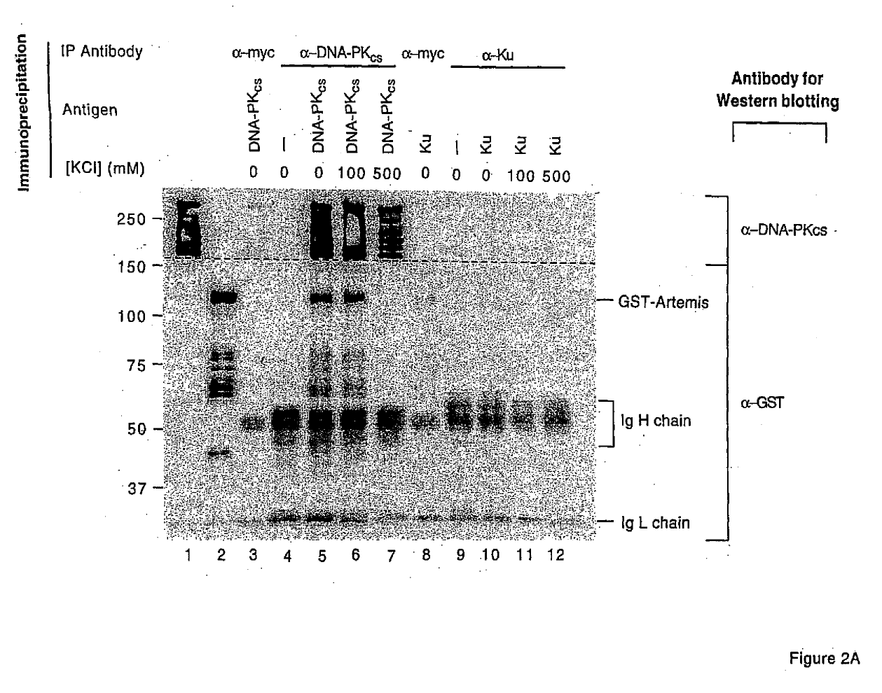

More specifically, and with reference to FIG. 1, the site-specific V(D)J recombination process occurs precisely at the end of each V, D or J gene segment (i.e., at the coding end) where it is bordered by recombination signal sequences (RSS, indicated by triangles in FIG. 1). Each RSS consists of conserved heptameter and nonamer motifs separated by 12- or 23-nucleotide “spacer” sequences and is designated 12-RSS or 23-RSS based on the spacer length (Gellert, 1997). Recombination events occurring within lymphoid cells require one 12-RSS and one 23-RSS; this feature is designated as the 12/23 rule. The recombination activating genes RAG-1 and RAG-2, along with HMG1 or HMG2, recognize and form a complex (the RAG complex) with the heptameter/nonamer recombination signal sequences. The RAG complex then uses the 3′-hydroxyl on each V, D), or J coding end as a nucleophile in a transesterification attack (vanGent et al., 1996) on the antiparallel DNA strand and endonucleolytically nicks the DNA 5′ of the heptameter precisely between the V, D, or J coding sequence and the RSS. As a result, two types of DNA ends are generated: blunt signal ends (which terminate in the RSS) and covalently sealed (hairpin) coding ends (which terminate in the V, D, or J element). After cleavage, the two signal ends are joined, producing a signal joint (FIG. 1). However, prior to joining the coding ends, the hairpins must be opened as discussed below.

After generation of the two hairpinned coding ends and the two signal ends, the four DNA ends appear to be held by the RAGS in a post-cleavage complex (Agrawal and Schatz, 1997; Hiom and Gellert, 1997). In order for the variable domain exon to be created, the V and J coding ends must each be released from their hairpin configuration and modified into a compatible configuration by unknown factors (designated by the question marks in FIG. 1) which include nuclease(s), template-dependent polymerase(s), as well as a template-independent polymerase (terminal deoxynucleotidyl transferase). One of the major unresolved questions in V(D)J recombination concerns how the hairpinned coding ends are opened. This question can be assayed using DNA oligonucleotide hairpins (termed free hairpins), or it can be assayed using RAG-generated hairpins. It has been shown that the RAG complex can open free hairpins (Besmer et al., 1998). However, this activity of the RAG complex is only substantial in manganese ion-containing buffers, and it is not observed in magnesium ion-containing buffers. Many nucleases have altered specificities when provided with Mn²⁺. Hence, these observations are interesting, but may not be physiologically relevant. There is also data suggesting that the RAG complex can open RAG-generated hairpins (Besmer et al., 1998; Shockett and Schatz, 1999). However, the efficiency of this process has been documented to be extremely low (Shockett and Schatz, 1999; K. Yu and M. Lieber, unpublished), causing uncertainty about its physiologic relevance. Moreover, this low level of hairpin opening is not dependent on DNA-PKcs or DNA-PK_{cs}. (K. Yu and M. Lieber, unpublished). Given the uncertainties about RAG hairpin opening in buffers containing magnesium ions, it remains unclear what enzyme opens the hairpins in V(D)J recombination.

Once the hairpins are opened, the joining phase of V(D)J recombination is carried out by the nonhomologous DNA end joining pathway (NHEJ; FIG. 1) (Lieber, 1999). The NHEJ pathway, which is responsible to join both the coding and the signal ends to form the coding joints and signal joints, is present in somatic cells of all multicellular eukaryotes, whereas the RAG complex is unique to lymphoid cells. It is the major DNA double-strand break repair pathway, and defects in this pathway result in sensitivity to DNA double-strand break agents (such as X-rays) in all somatic cells and failure to complete V(D)J recombination in lymphoid cells (van Gent et al., 2001). X-ray sensitivity, genetic, and biochemical studies have permitted the identification of several key proteins in the NHEJ pathway (Wood et al., 2001). Ku and the 4,127 amino acid (469 kDa) DNA-dependent protein kinase catalytic subunit (DNA-PK_{cs}) each can bind independently to DNA ends (Hammarsten and Chu, 1998; West et al., 1998; Yaneva et al., 1997). However, upon Ku binding to a DNA end, Ku improves the affinity of DNA-PK_{cs} for the DNA end by 100-fold (West et al., 1998). The crystal structure for Ku (Walker et al., 2001) and the lower resolution structures for DNA-PK_{cs} (Chiu et al., 1998; Leuther et al., 1999) are consistent with models in which each protein can bind at the single-strand to double-strand transitions in DNA. Recently, Ku has been reported to associate with inositol hexakisphosphate (IP₆) in vitro (Ma and Lieber, 2002), while IP₆ was shown to be able to stimulate DNA end joining in a cell free system (Hanakahi et al., 2000). Thus, the potential role of Ku in hairpin opening might be revealed by the addition of IP₆.

Although DNA-PK_{cs} is a DNA end-dependent serine/threonine protein kinase, and although in vitro it can phosphorylate many polypeptides, its relevant phosphorylation targets in V(D)J recombination and in NHEJ have remained undefined (Anderson and Carter, 1996). For example, DNA-PK_{cs} can phosphorylate RAG-1 and RAG-2 in vitro (R. West, K. Yu and M. Lieber, unpublished), but mutation of all of the DNA-PK_{cs} consensus phosphorylation sites in the RAG-1 and RAG-2 proteins has no discernable effect on V(D)J recombination, raising further doubts that RAG-1 and RAG-2 possess hairpin opening activity (Lin et al., 1999). The precise role of Ku and DNA-PK_{cs} in V(D)J recombination and in NHEJ has not been entirely clear) although in their absence the hairpinned coding ends of V(D)J recombination remain unopened (Roth et al., 1992; Zhu et al., 1996). Since neither Ku nor DNA-PK_{cs} possess documented enzymatic activity on nucleic acid substrates, it has been hypothesized that DNA-PK_{cs} either recruits or affects the hairpin opening activity by phosphorylation (Blunt et al., 1995).

The two DNA ends generated at pathologic double-strand DNA breaks are rarely compatible. In the physiologic dsDNA breaks of V(D)J recombination, the coding end hairpins are suspected to be opened preferentially 3′ to the loop or “tip” of the hairpin (Schlissel, 1998), resulting in only a minority of ends with terminal microhomology. The nucleases involved in trimming the ends and the polymerases involved in filling-in any gaps in NHEJ have yet to be definitively identified. In S. cerevisiae, there is genetic evidence supporting the role of polymerase β in filling-in a subset of the gaps and of FEN-1 in trimming some of the 5′ flaps (Wilson and Lieber, 1999; Wu et al., 1999). The necessary nucleases and polymerases involved in NHEJ of multicellular eukaryotes have not been identified (designated as question marks in FIG. 1).

The best understood phase of the NHEJ pathway is the ligation step, where it is clear that the ligase is DNA ligase IV in yeast, mice, humans, and presumably in all eukaryotic organisms, including plants (Barnes et al., 1998; Gao et al., 1998; Grawunder et al., 1997; Grawunder et al., 1998; Schar et al., 1997; Teo and Jackson, 1997; Wilson et al., 1997). XRCC4 is a polypeptide that forms a heteromultimer with DNA ligase IV, is required in vivo, and is stabilizing and stimulatory for DNA ligase IV function (Grawunder et al., 1997; Modesti et al., 1999).

RAG mutations and NHEJ component null mutations have been found to result in a severe combined immune deficiency (SCID) (Schwarz et al., 1996; Vanasse et al., 1999; Villa et al., 2001). The mutations in the NHEJ pathway also result in sensitivity to agents that cause double-strand DNA breaks, such as X-rays and bleomycin. The most recently identified gene of which mutation results in X-ray sensitivity and in SCID is called Artemis (Moshous et al., 2001). The putative protein encoded by the Artemis gene only has limited homology to the SNM1 protein of S. cerevisiae and mouse, the absence of which results in sensitivity to DNA interstrand cross-linking agents (Dronkert et al., 2000; Henriques and Moustacchi, 1980). Human cells deficient for the Artemis protein have the same V(D)J recombination phenotype as murine DNA-PK_{cs} mutants (Bosma and Carroll, 1991; Hendrickson et al., 1991; Lieber et al., 1988; Moshous et al., 2001; Nicolas et al., 1998; Schuler et al., 1986). That is, signal joint formation occurs at normal or near normal levels, whereas coding joint formation is reduced over 1000-fold (Harrington et al., 1992; Moshous et al., 2001). No enzymatic activity has thus far been reported for Artemis.

### SUMMARY OF THE INVENTION

One aspect of this invention is based on the discovery that Artemis alone demonstrates single-strand specific 5′ to 3′ exonucleolytic activity. More specifically, it was observed that Artemis exonucleolytically cleaves specific single stranded nucleotides, such as 5′ single-stranded overhangs linked to double-stranded DNA and mismatched nucleotides at the end of a duplex DNA. Accordingly, one aspect of this invention provides an exonucleolytic composition consisting essentially of Artemis. It was further discovered that the exonucleolytic activity of Artemis is more effective in buffers containing magnesium ions. Thus, another aspect of this invention provides an exonucleolytic composition consisting essentially of Artemis and a magnesium ion-containing buffer.

This invention further provides a method of exonucleolytically cleaving a single-stranded nucleotide, said method comprising contacting said nucleotide with a composition consisting essentially of Artemis or a composition consisting essentially of Artemis a magnesium ion-containing buffer under conditions that allow Artemis to cleave said nucleotide. The single-stranded nucleotide may be RNA or DNA, and further may be a 5′ nucleotide overhang or a sequence of mismatched nucleotides at one or both ends of a double-stranded DNA.

This invention further provides assays based on the ability of the Artemis to exonucleolytically cleave nucleotides in a site-specific and structure-specific manner. For example, one embodiment of this invention provides an assay for analyzing a branched nucleic acid such as a nucleic acid containing a 5′ nucleotide overhang or a double-stranded DNA suspected of containing mismatched sequences at one or both ends. Thus, one assay of this invention comprises contacting said nucleotide with an exonucleolytic composition consisting essentially of Artemis or an exonucleolytic composition consisting essentially of Artemis and a magnesium ion-containing buffer under conditions that allow Artemis to cleave said nucleotide, and analyzing the resulting composition by gel electrophoresis or a variety of substituted methods known in the art such as fluorescence or radioactivity-based methods to determine if said nucleic acid was cleaved.

Another aspect of this invention is based on the discovery of the relationship between the Artemis and a component of the NHEJ pathway, i.e., the catalytic subunit of the DNA-dependent protein kinase (DNA-PK_{cs}). More specifically, this invention demonstrates that Artemis and DNA-PK_{cs} form a complex both in vitro and in vivo in the absence of DNA, and that the activity of Artemis is regulated by DNA-PK_{cs}. For example, it was discovered that upon complex formation with DNA-PK_{cs}, Artemis switches from being an exonuclease to an endonuclease, and the endonucleolytic activity requires that Artemis remain complexed to DNA-PK_{cs}. It was further observed that DNA-PK_{cs} efficiently phosphorylates Artemis and thus regulates the enzymatic activity of Artemis in a process that is ATP-dependent. Accordingly, another aspect of this invention provides an endonucleolytic composition comprising a complex of Artemis and DNA-PK_{cs}.

It was observed that the Artemis:DNA-PK_{cs} complex is able to endonucleolytically cleaves 5′ as well as 3′ single-stranded overhangs. Thus, a further aspect of this invention comprises a method of endonucleolytically cleaving a 5′ or 3′ nucleotide overhang of a double-stranded DNA, comprising combining said DNA with a composition comprising an Artemis:DNA-PK_{cs} complex under conditions that allow said Artemis:DNA-PK_{cs} complex to endonucleolytically said overhang. In one embodiment, the composition further contains a phosphorylating agent. In another embodiment, the composition further comprises a magnesium ion-containing buffer. This method can further be used as an assay for analyzing a nucleic acid suspected of containing a 5′ or 3′ overhang, wherein after subjecting the nucleic acid to the endonucleolytic conditions, the resulting composition is analyzed to determine if endonucleolytic cleavage occurred.

This invention is further based on the discovery that although Artemis alone has no effect on hairpins, the Artemis:DNA-PK_{cs} complex is able to endonucleolytically cleave and open hairpins, including hairpins generated by the RAG complex (RAG-1, RAG-2, and HMG1 or HMG2). It was further discovered that both the physical presence of DNA-PK_{cs} in a complex with Artemis, as well as the kinase activity of DNA-PK_{cs} is required for this effect.

Accordingly) another aspect of this invention comprises a method of opening a double-stranded nucleic acid having a hairpin configuration comprising a single-stranded loop, said method comprising combining said nucleic acid with a composition comprising an Artemis:DNA-PK_{cs} complex under conditions that allow said Artemis:DNA-PK_{cs} complex to cleave said nucleotide, wherein the cleavage occurs at the beginning of said loop or at a position within said loop. In one embodiment, the conditions include adding a magnesium ion-containing buffer. In another embodiment, the conditions include adding a phosphorylating agent. This invention therefore provides the first eukaryotic hairpin opening activity by a nuclease that functions efficiently in magnesium ion-containing buffers.

This invention further provides methods for developing assays based on the ability of the Artemis:DNA-PK_{cs} complex to cleave nucleotides in a site-specific and structure-specific manner, and the assays developed therefrom. Such assays may be useful for the diagnosis of infectious diseases caused by viruses, bacteria, fungi, inherited mutations, or acquired mutations such as tumors.

Accordingly, another aspect of this invention comprises a method of analyzing a nucleic acid suspected of containing a hairpin motif, said method comprising providing a composition comprising an Artemis:DNA-PKcs complex; contacting said complex with said nucleic acid under conditions that allow said complex to cleave and open nucleic acid hairpins; and analyzing said nucleic acid by gel electrophoresis or a variety of substituted methods known in the art such as fluorescence or radioactivity-based methods.

Artemis is a natural enzyme in every vertebrate cell, including humans. As a result of the discovery herein of the role of Artemis in DNA repair pathways, this invention further provides methods for the identification and development of therapeutic compounds that inhibit Artemis, such as compounds for the treatment of cancers.

For example, in accordance with another aspect of the present invention, there is provided a method for identifying a compound capable of inhibiting Artemis protein activity, the method comprising:
(a) preparing a reaction mixture by combining Artemis protein with or without DNA-PKcs and with at least one test compound under conditions permissive for the activity of Artemis for a predetermined amount of time;
(b) assessing the activity of Artemis with or without DNA-PKcs and in the presence of the test compound after said predetermined length of time; and
(c) comparing the activity of Artemis with or without DNA-PKcs and in the presence of the test compound with the activity of Artemis with or without DNA-PKcs and in the absence of the test compound, wherein a decrease in the activity of Artemis in the presence of the test compound is indicative of a compound that acts as an inhibitor of Artemis.

In one embodiment, the activity is measured after said predetermined length of time by contacting the reaction mixture with a double-stranded DNA comprising a terminal single-stranded nucleotide, and determining whether said Artemis exonucleolytically cleaves said single-stranded nucleotide.

Yet another aspect of this invention provides a method of ameliorating a condition caused by the activity of Artemis in a patient, comprising administering to said patient an amount of a compound effective to inhibit the activity of Artemis. Such compounds may be useful in treating cancers such as acute lymphoblastic leukemia based on the role of Artemis in opening hairpins in lymphoid cells.

A further aspect of this invention contemplates a method of enhancing cancer therapy in a patient, comprising delivering a compound that inhibit Artemis to cancerous cells in said patient, followed by administering one or more traditional cancer therapies to said patient.

This invention further provides assays for diagnosing conditions caused by abnormal or altered levels of Artemis. For example, with respect to cancer, the presence of a relatively high amount of Artemis in biopsied tissue from an individual may indicate a predisposition for the development of the disease, or may provide a means for detecting the disease prior to the appearance of actual clinical symptoms.

This invention is further based on the discovery that the Artemis protein structure comprises a beta-lactamase fold that is necessary for its function. This structural fold is the same as that found in the enzyme beta-lactamase, a protein that confers penicillin-resistance upon penicillin-resistant bacteria. Accordingly this invention further contemplates a method of identifying a compound that inhibits the activity of Artemis, comprising providing a compound known to inhibit beta-lactamase, contacting said compound with Artemis protein, and determining if said activity is inhibited.

If desired, Artemis protein used in the methods and assays of this invention may be purified from bacterial sources or, preferably, are produced by recombinant DNA techniques, since the gene coding for Artemis is known. Accordingly, this invention also provides a method of producing recombinant Artemis. In one embodiment, the method produces a fusion protein comprising Artemis linked directly or indirectly to an affinity tag. The presence of the affinity tag is useful, for example for purifying Artemis. In one embodiment, the fusion protein is designed so that the affinity tag can be cleaved from Artemis.

Accordingly, another embodiment of this invention provides a method of purifying Artemis, wherein the method comprises expressing the Artemis gene as a fusion protein comprising a recombinant Artemis linked directly or indirectly to an affinity tag; contacting said fusion protein with a matrix comprising a compound that binds said affinity tag under conditions that allow said compound to bind said affinity tag, and recovering said fusion protein to provide a purified fusion protein. In another embodiment, the Artemis gene is expressed as a fusion protein comprising the Artemis protein linked to DNA-PK_{cs}. In this embodiment, the fusion protein may also comprise an affinity tag.

Additional advantages and features of this invention shall be set forth in part in the description that follows, and in part will become apparent to those skilled in the art upon examination of the following specification or may be learned by the practice of the invention. The features and advantages of the invention may be realized and attained by means of the instrumentalities, combinations, and methods particularly pointed out in the appended claims.

### BRIEF DESCRIPTION OF THE DRAWINGS

The accompanying drawings, which are incorporated in and form a part of the specification, illustrate non-limiting embodiments of the present invention, and together with the description serve to explain the principles of the invention.

### In the Figures:

FIG. 1 illustrates the V(D)J recombination pathway, showing the RAG-dependent and NHEJ phases. V and J represent the variable and joining elements (subexons), respectively, and the 12-RSS and 23-RSS recombination signal sequences are indicated by triangles.

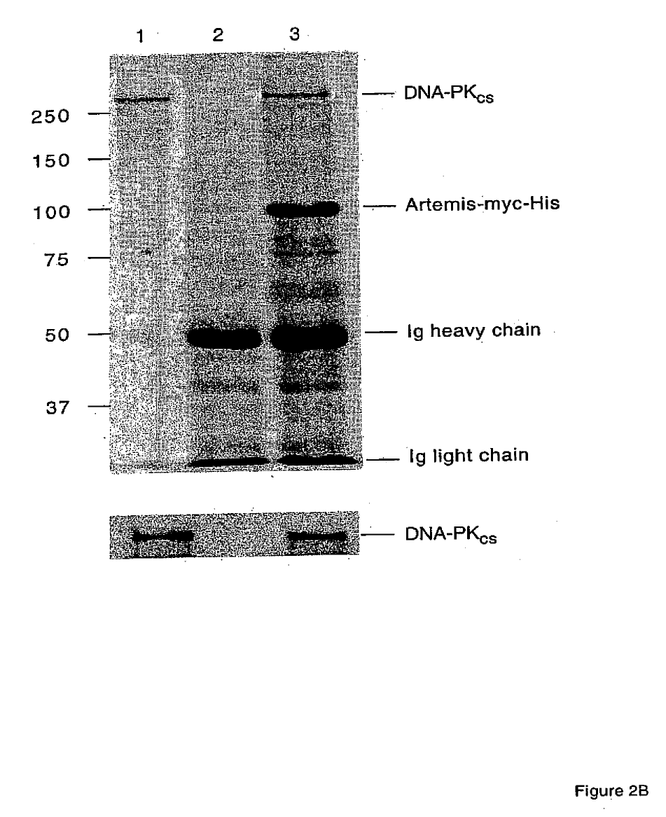

FIG. 2A is an image of an 8% SDS-polyacrylamide gel from a Western blot analysis of an Artemis immunobead pull-down assay using immobilized GST-Artemis.

FIG. 2B is an image of an 8% SDS-polyacrylamide gel from a Western blotting analysis using immobilized Ku.

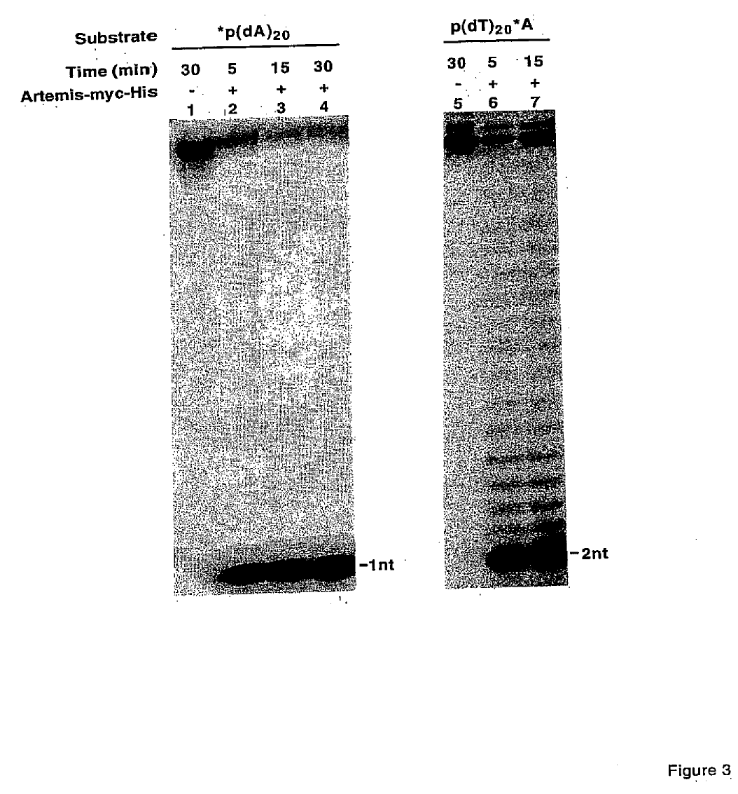

FIG. 3 is an image of a gel in which a single-stranded poly dA 20-mer was labeled with T4 polynucleotide kinase (T4 PNK) and incubated for the specified times with Artemis-myc-His (lanes 2 to 4). A poly dT 20-mer 3′ labeled with terminal deoxynucleotidyl transferase and [α-32P] dideoxyadenosine triphosphate is shown in lanes 5 to 7.

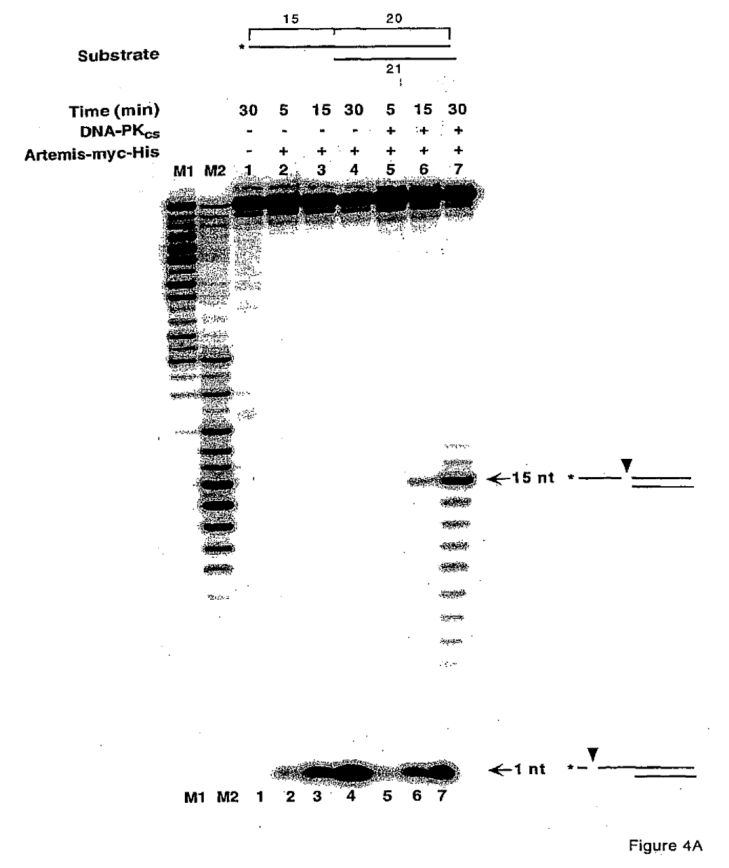

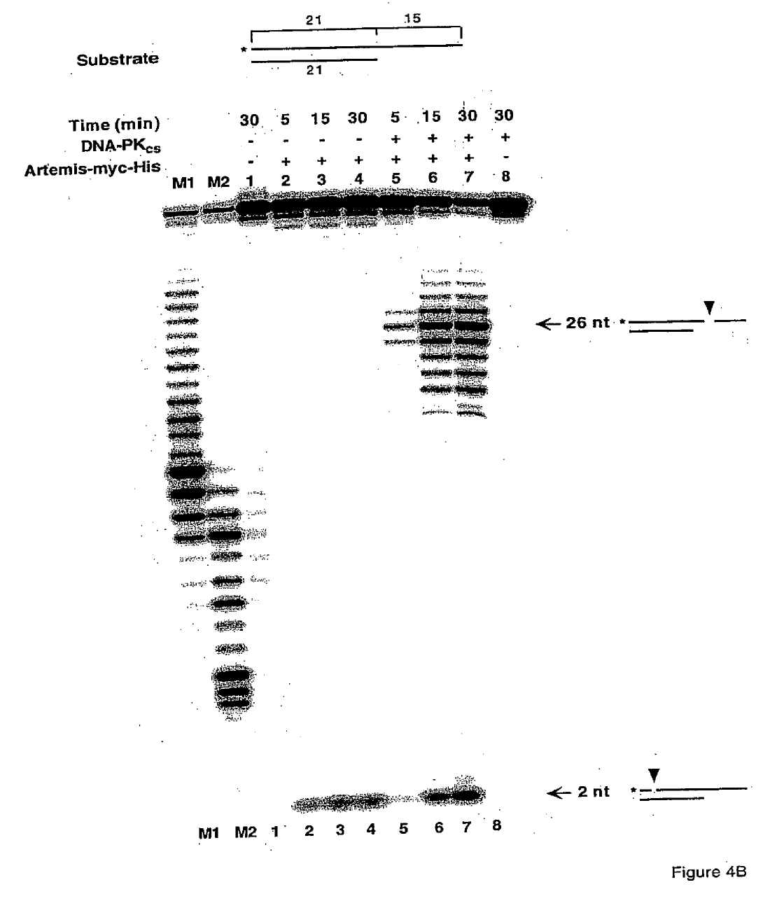

FIG. 4 A is an image of a gel in which a double-stranded oligonucleotide with a 5′ 15 nucleotide overhang labeled with T4 PNK on the long strand (as indicated by the asterisk) was degraded by Artemis-myc-His alone or Artemis and DNA-PK_{cs}.

FIG. 4B is an image of a gel in which a double-stranded oligonucleotide with a 3′ 15 nucleotide (thymidine) overhang labeled with T4 PNK on the long strand (as indicated by the asterisk) was degraded by Artemis-myc-His alone or Artemis and DNA-PK_{cs} is shown.

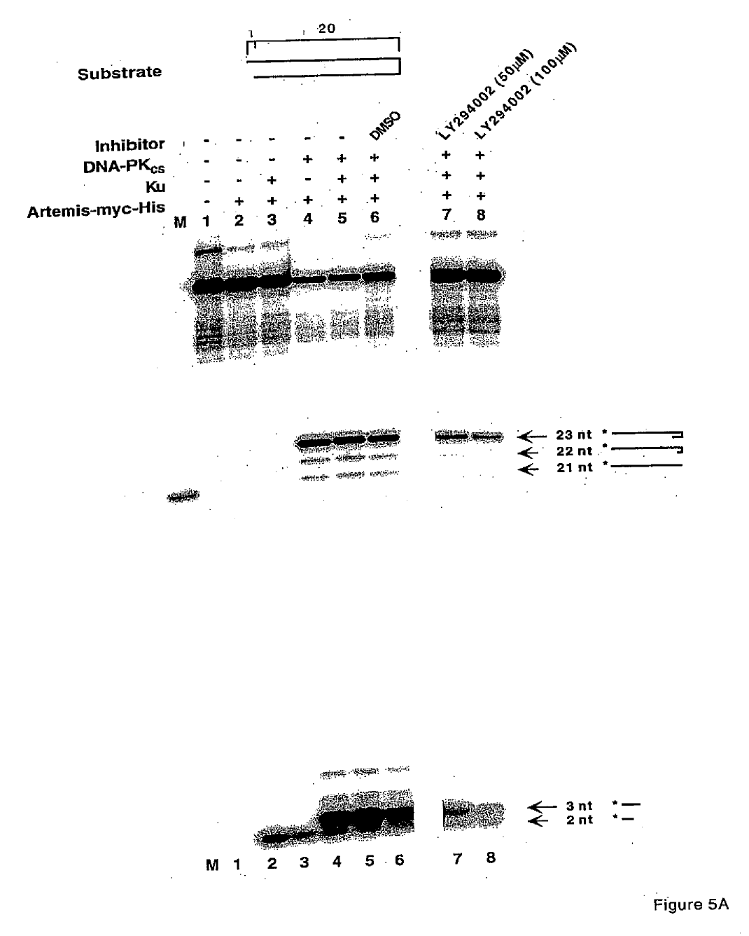

FIG. 5A is an image of a gel in which a 20 bp hairpin (D_{FL16.1}) with a 1-nucleotide 5′ overhang labeled at the 5′ end with T4 PNK was used as the substrate. In reactions with inhibitors, DNA-PK was either mock treated with DMSO (lane 6) or treated with LY294002 at 50 μM (lane 7) and 100 mM (lane 8) first, and then the substrate was added.

FIG. 5B is an image of a gel in which a 20 bp hairpin (D_{FL16.1}) with a 1-nucleotide 5′ overhang labeled at the 5′ end with T4 PNK was subject to a hairpin opening assay in presence of 1 mM of ATP (lane 4) or 1 mM of ATP analogs ATP-γ-S (lane 5) or AMP-PNP (lane 6).

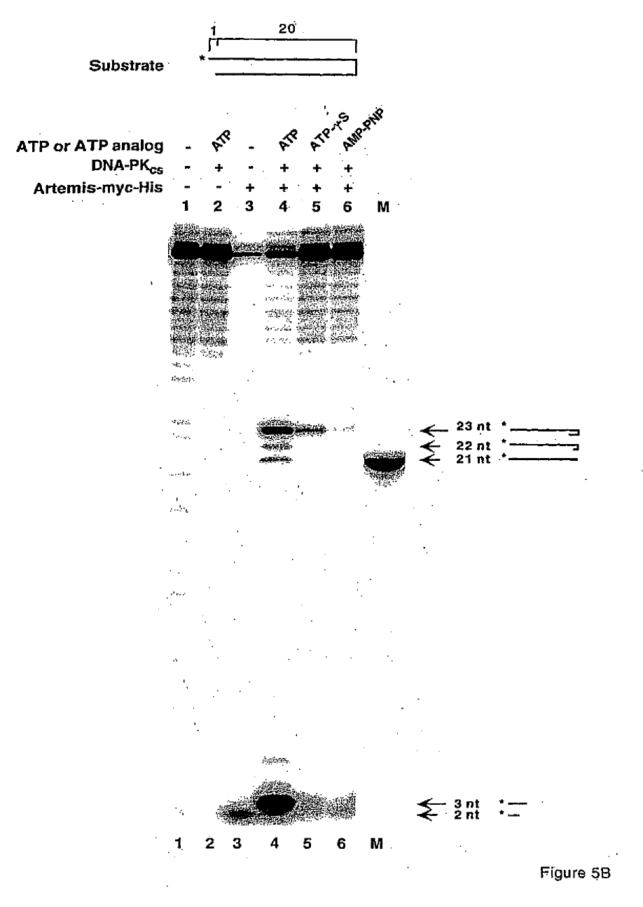

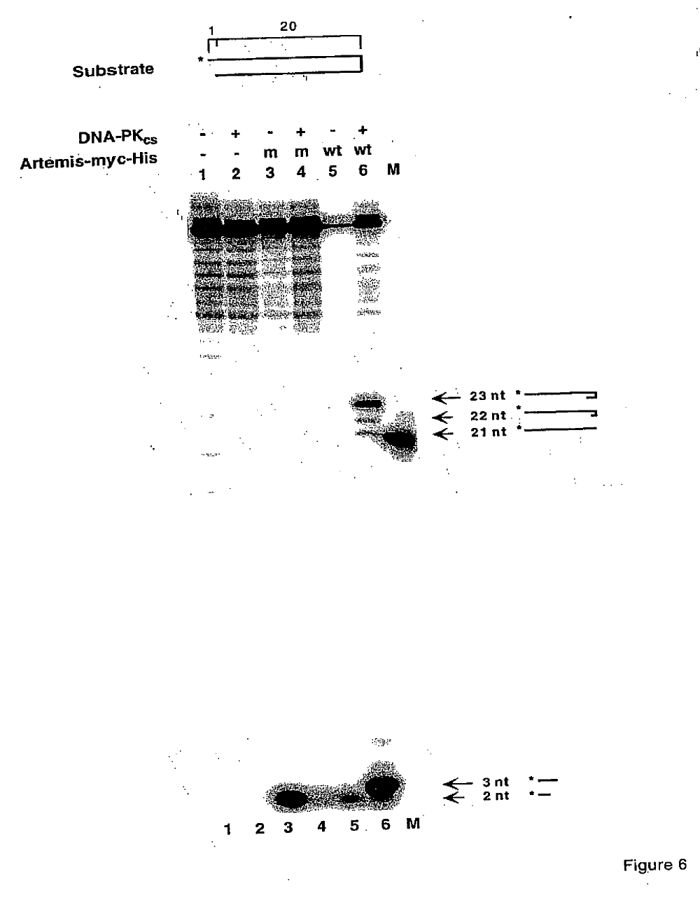

FIG. 6 is an image of a gel in which a 20 bp hairpin (D_{FL16.1}) with a 1-nucleotide 5′ overhang labeled at the 5′ end with T4 PNK was used as the substrate.

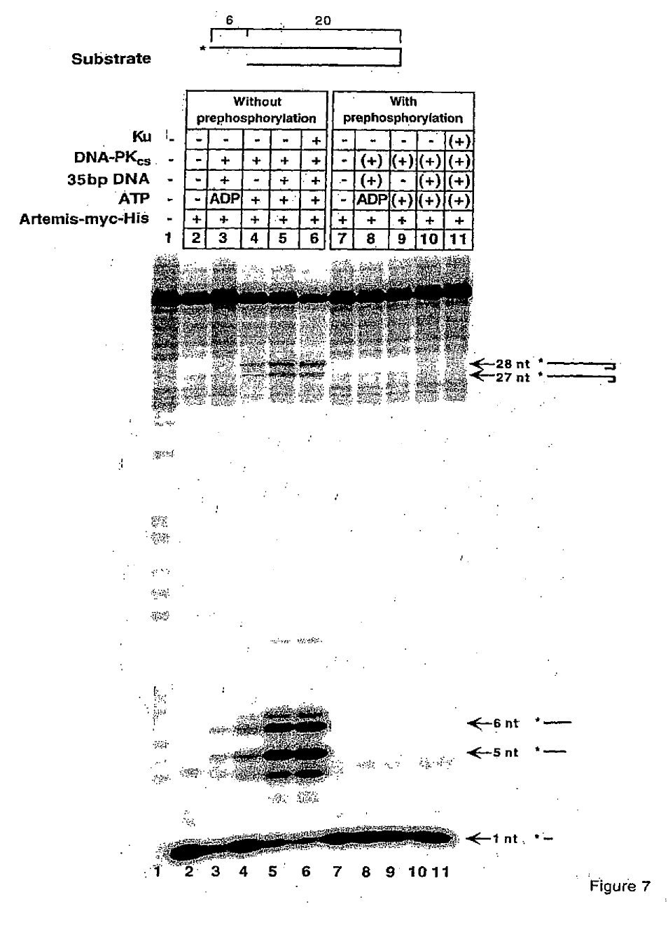

FIG. 7 is an image of a gel in which a 20 bp artificial hairpin with a 6-nucleotide 5′ overhang labeled with T4 PNK was used as the substrate.

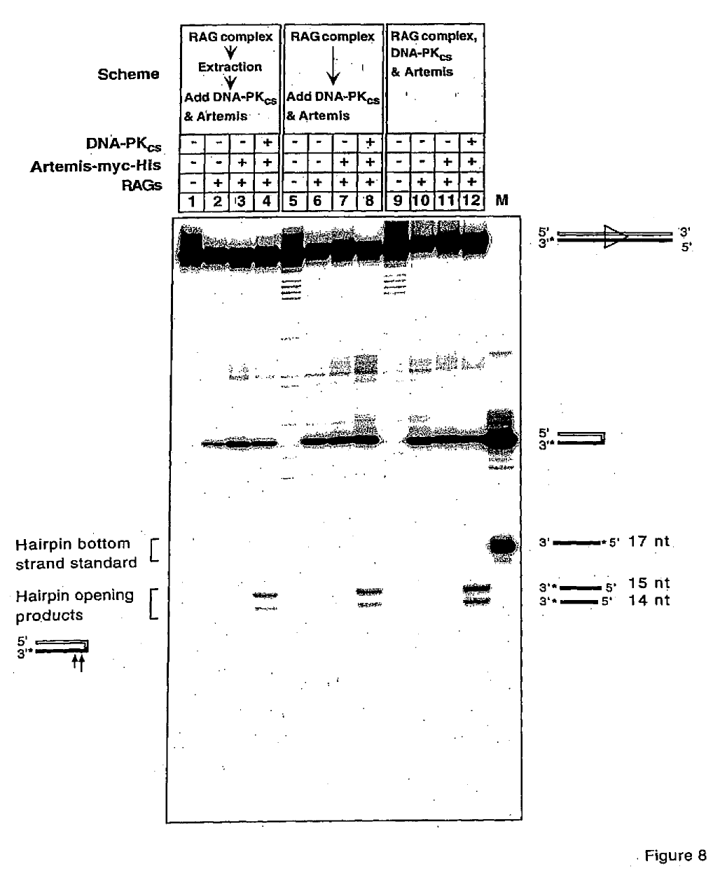

FIG. 8 is a gel of a hairpin-formation and opening experiment carried out with three different configurations using a RAG-generated hairpin.

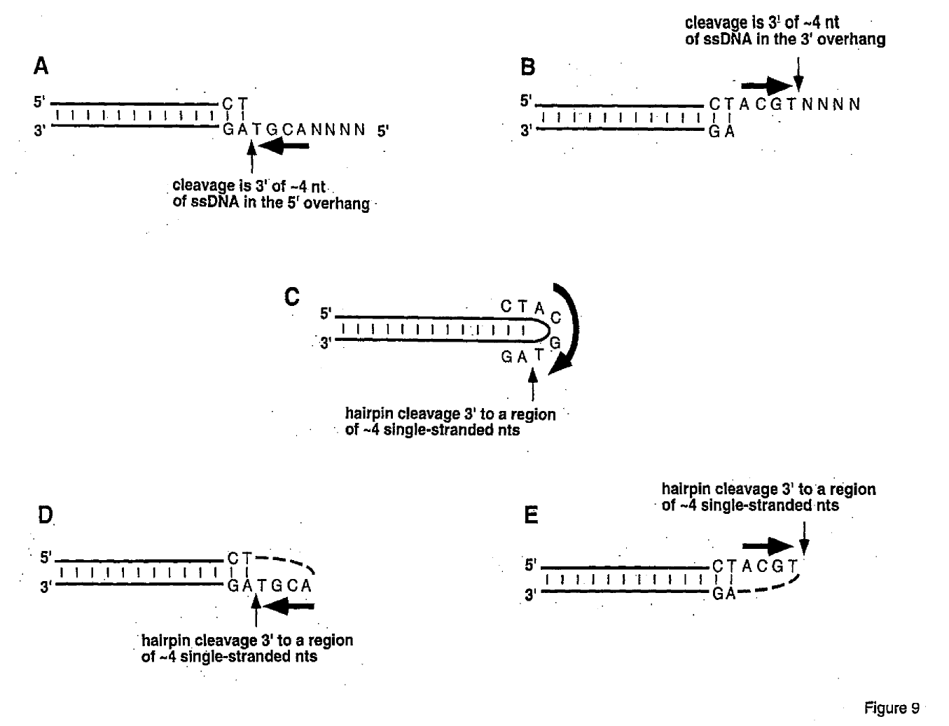

FIGS. 9A and 9B are schematic structures of a dsDNA with a 3′ and a 5′ overhang, respectively. Thin arrows mark the major cleavage sites observed in the assay described in FIGS. 4A and 4B on similar DNA structures, respectively. N represents any nucleotide in the overhangs. The thick arrow depicts the hypothesized recognition region by Artemis.

FIG. 9C is a schematic structure of a hairpin with a D_{FL16.1} coding end sequence (the substrate for FIGS. 5A, 5B and 6, in which only the terminal 8-nucleotide strand is shown). The major cleavage position by the Artemis:DNA-PK_{cs} complex (2 nucleotides 3′ to the hairpin tip or +2 position) is marked by the thin arrow. The thick arrow depicts the hypothesized recognition region by Artemis.

FIGS. 9D and 9E are schematic structures of a hairpin with a D_{FL16.1} coding end sequence with emphasis on the structural similarity to a dsDNA with a 5′ and a 3′ overhang, respectively. Dashed lines represent the artificially stretched phosphodiester bonds at the −2 and +2 positions, respectively. The thick arrow depicts the hypothesized recognition region by Artemis.

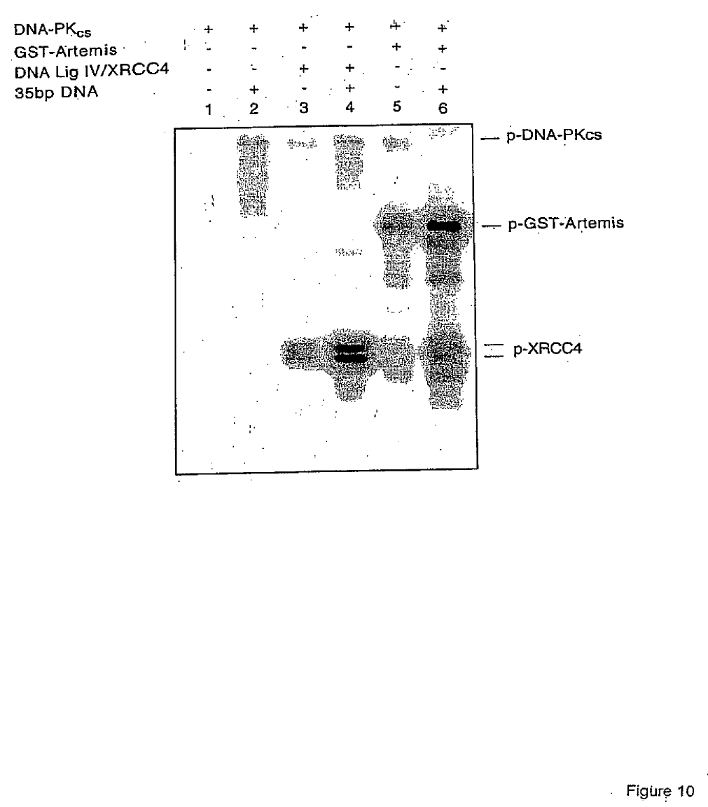

FIG. 10 is an autoradiogram of a DNA-PK kinase assay in which a 35 bp DNA was used as the DNA-PKcs cofactor.

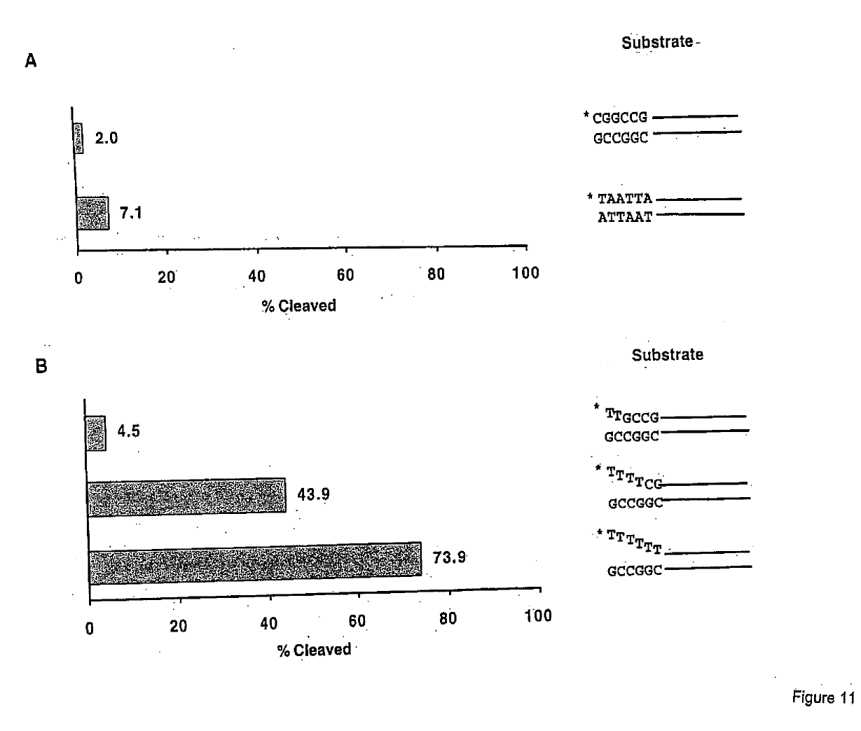

FIG. 11A is a bar graph showing the percentage of cleaved substrate out of the total input substrate labeled with T4 PNK on one strand as indicated by the asterisks after double-stranded oligonucleotides with GC- or AT-rich end were incubated with Artemis-myc-His.

FIG. 11B is a bar graph showing percentage of the cleaved substrate out of the total input substrate labeled with T4 PNK on one strand as indicated by the asterisks after double-stranded oligonucleotides with GC- or AT-rich end were incubated with Artemis-myc-His. In this example, the DNA have terminal mismatches of different lengths.

### DETAILED DESCRIPTION OF THE INVENTION

The findings presented herein provide insights into several previously unanswered questions in V(D)J recombination and in NHEJ. For example, this invention describes the nuclease activity of the Artemis protein alone. Further, this invention describes the physiologic phosphorylation target of DNA-PK_{cs} (i.e., Artemis) in the context of V(D)J recombination. The results presented herein also explain why the absence of DNA-PK_{cs} results in the failure to open hairpinned coding ends, despite the fact that DNA-PK_{cs} has no nuclease activity of its own, nor can DNA-PK_{cs} confer efficient hairpin opening activity on the RAG complex. In addition, by demonstrating that Artemis is the component in the Artemis:DNA-PK_{cs} complex having hairpin opening activity, this invention describes its role in general NHEJ.

Methods of cloning and expressing the Artemis gene are fully described by Moshous et al. (2001), which reference is incorporated herein by reference. In addition to the Artemis protein, analog of Artemis may be used in the compositions and methods of this invention, provided that the analog comprises a protein having nuclease activity that is sufficiently similar to DNA-PK_{cs}. Such analogs will be considered as equivalents of Artemis for purposes of this invention. As used herein, an “analog” may include any homologue of the Artemis protein, such as a protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol).

More specifically, one aspect of this invention is based on the discovery that Artemis protein alone demonstrates single-strand specific 5′ to 3′ exonucleolytic activity, and has no endonuclease activity on dsDNA. More specifically, it was observed that Artemis exonucleolytically cleaves 5′ monophosphates from single stranded nucleotides, and this exonuclease activity appears to be processive rather than distributive. Examples of such single-stranded nucleotides include, but are not limited to, 5′ single-stranded overhangs linked to double-stranded DNA, and mismatched nucleotides at the end of a duplex DNA. The exonucleolytic activity of Artemis was observed to increase markedly on substrates with an increasing number of terminal mismatches.

Accordingly, one aspect of this invention provides an exonucleolytic composition consisting essentially of Artemis. As used herein, an “exonucleolytic composition” or an “exonuclease” is a composition or enzyme, respectively, that cleaves nucleotides one at a time from an end of a polynucleotide chain. The exonucleolytic activity was observed to be more effective in buffers containing magnesium ions, and is inactive in buffers containing manganese or zinc ions. Accordingly, this invention further provides an exonucleolytic composition consisting essentially of Artemis and magnesium ions.

It was observed that the 5′ exonuclease activity of Artemis is strongly dependent on the presence of a 5′ phosphate on the single-stranded nucleotide, and showed substantially equivalent activity on RNA and DNA.

The exonucleolytic activity of Artemis alone may be regarded as unregulated, and clearly this activity is insufficient for general NHEJ (and for V(D)J recombination) because DNA-PK_{cs} mutants are sensitive to ionizing radiation (Hendrickson et al., 1991). The orientational polarity of the Artemis:DNA-PK_{cs} complex on 5 overhangs may be a reflection of the polarity of Artemis alone as a 5′ to 3′ exonuclease. Further studies will be needed to test the various aspects of this model.

This invention further provides a method of exonucleolytically cleaving a single-stranded nucleotide, said method comprising contacting said nucleotide with an exonucleolytic composition consisting essentially of Artemis or an exonucleolytic composition consisting essentially of Artemis and a magnesium ion-containing buffer under conditions that allow Artemis to cleave said nucleotide. The single-stranded nucleotide may be, for example, a 5′ nucleotide overhang or a sequence of mismatched nucleotides at one or both ends of a double-stranded DNA.

This method may further be used as an assay for analyzing nucleic acids such as branched DNA. For example, one embodiment of this invention provides an assay for analyzing a nucleic acid suspected of containing a branched nucleic acid. The assay comprises contacting said nucleotide with an exonucleolytic composition consisting essentially of Artemis or an exonucleolytic composition consisting essentially of Artemis and a magnesium ion-containing buffer under conditions that allow Artemis to cleave said nucleotide, and analyzing the resulting composition by gel electrophoresis or any of a variety of substituted methods such as fluorescence or radioactivity-based methods to determine if said nucleic acid was cleaved. In one embodiment, the composition may be analyzed by comparing the resulting composition after the assay with a sample of the nucleic acid that was not subjected to the assay.

The term “branched DNA” as used herein refers to a double-stranded DNA comprising, for example, a 5′ nucleotide overhang, or a sequence of mismatched nucleotides at one or both ends of the double-stranded DNA. “Branched DNA” also refers to structures including, but not limited to, pseudo-k nucleotides, strand displacement structures, and recombination intermediates.

As used herein, the term “nucleotide” means a deoxyribonucleotide, a ribonucleotide, or any nucleotide analogue. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH₂, NHR, NR₂, or CN. Nucleotides can also include non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

The term “oligonucleotide” refers to an oligomer or polymer of ribonucleic acid (RNA) and/or deoxyribonucleic acid (DNA) or mimetics thereof, as well as oligonucleotides having non-naturally-occurring portions which function similarly.

Another aspect of this invention is based on the discovery of the relationship between the Artemis protein and a component of the NHEJ pathway, i.e., the catalytic subunit of the DNA-dependent protein kinase (DNA-PK_{cs}). More specifically, this invention demonstrates that Artemis and DNA-PK_{cs} form a complex both in vitro and in vivo in the absence of DNA or DNA termini, and that the activity of Artemis is regulated by DNA-PK_{cs}.

As discussed in detail in the Examples, it has been shown herein that while Artemis alone is a 5′ to 3′ single-strand exonuclease, the Artemis:DNA-PK_{cs} complex acts as an endonucleolytic enzyme. For example, it was observed that the Artemis:DNA-PK_{cs} complex is an overhang endonuclease and a hairpin endonuclease, and this activity is dependent on DNA ends. DNA-PK_{cs} not only forms a physical complex with Artemis, but it is also able to efficiently phosphorylates Artemis. DNA-PK_{cs} thus regulates the enzymatic activity of Artemis in a process that is ATP-dependent. The Artemis:DNA-PK_{cs} complex of this invention is stable under physiologic ionic strength and does not rely on DNA termini or Ku for stability. These results imply that the Artemis:DNA-PK_{cs} nuclease complex would be ideally responsive to pathologic dsDNA breaks.

As used herein, the terms “endonuclease” or “endonucleolytic composition” refers to an enzyme or a composition, respectively, that breaks the internal phosphodiester bonds in a DNA molecule.

This invention further shows that it is likely that Artemis and DNA-PK_{cs} function as a complex. For example, pretreatment of Artemis with DNA-PK_{cs} and ATP was not sufficient to confer overhang cleavage and hairpin opening activity on Artemis. Rather, DNA-PK_{cs} must remain present, even after the phosphorylation, for efficient hairpin opening. Since DNA-PK_{cs} alone did not show nuclease activities, the nucleolytic active site probably resides in Artemis, and the failure of a point mutant of Artemis to open hairpins strongly supports this argument. The fact that the regulation of Artemis endonucleolytic activity by Artemis:DNA-PK_{cs} is ATP-dependent indicates that the kinase activity of DNA-PK_{cs} is necessary. It remains to be determined whether the key protein phosphorylation events within the Artemis:DNA-PK_{cs} complex are DNA-PK_{cs} phosphorylation of itself, of Artemis, or both.

With DNA-PK_{cs} present, Artemis was observed to generate a series of endonucleolytic cleavages internal to the 5′ end of a nucleotide having a 5′ single-stranded overhang. In addition, DNA-PK_{cs} enables Artemis to cleave 3′ single-stranded overhangs. In certain cases, the complex demonstrated a preference for cleavage at the single-strand/double-strand junction of the nucleotide. In other cases, cleavage occurred at a position at least 1-10 nucleotides from the junction, depending on the length of the overhang.

Accordingly, another aspect of this invention provides an endonucleolytic composition comprising a complex of Artemis and DNA-PK_{cs}. DNA-PK_{cs} is an enzyme of 4,127 amino acids with an approximate molecular weight of 470 kDa. The amino acid sequence of DNA-PK_{cs} is fully described in Blunt et al. (1995), which is specifically incorporated herein by reference. In one embodiment, an analog of DNA-PK_{cs} may be used in the compositions of this invention. As used herein, an “analog of DNA-PK_{cs}” may include any homologue of the DNA-PK_{cs} protein, such as a protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) provided that the homologue comprises a protein having nuclease activity that is sufficiently similar to DNA-PK_{cs}. Such analogs will be considered as equivalents of DNA-PK_{cs} for purposes of this invention.

This invention further provides a method of endonucleolytically cleaving a 5′ or 3′ single-stranded overhang of a double-stranded DNA, comprising combining said DNA with a composition comprising an Artemis:DNA-PK_{cs} complex under conditions that allow said Artemis:DNA-PK_{cs} complex to endonucleolytically said overhang. The Artemis:DNA-PK_{cs} complex may be useful, for example, in processing DNA ends by endonucleolytically cleaving overhangs resulting from DNA double strand breaks before they are ligated.

It was also observed that the enzymatic activity of Artemis is dependent of the presence of a phosphorylating agent such as ATP or any other high energy phosphate compound. Thus, in accordance with another embodiment of this invention, the composition further comprises a phosphorylating agent. Further, the endonucleolytic activity of this composition was more effective when it contained a magnesium ion-containing buffer. Thus, in another embodiment, the composition further comprises magnesium ions.

In yet another embodiment, the composition comprises an Artemis:DNA-PK_{cs} complex, wherein the Artemis is linked directly or indirectly to an affinity tag, as described below.

The Artemis:DNA-PK_{cs} complex is also able to open hairpins, including hairpins that are generated by the RAG complex. The position of the hairpin opening varied, but a 3′ overhang was preferentially generated at the opened end. In one example the complex was able to cleave a hairpin generated from a RAG complex comprising a 12-nucleotide recombination signal sequence/23-nucleotide recombination signal sequence substrate pair in buffers containing Mg²⁺ and without removal of the RAG complex. These findings provide compelling evidence that the hairpin opening in V(D)J recombination and overhang processing in NHEJ are conducted by the Artemis: DNA-PK_{cs} complex.

Accordingly, another aspect of this invention provides a method of opening a double-stranded nucleic acid having a hairpin configuration comprising a single-stranded loop, said method comprising combining said nucleic acid with a composition comprising an Artemis:DNA-PK_{cs} complex under conditions that allow said Artemis:DNA-PK_{cs} complex to cleave said nucleotide, wherein the cleavage occurs at the beginning of or at a position within said single-stranded loop.

As used herein, the term “hairpin” refers to a nucleotide sequence that contains a double-stranded stem segment formed by two nucleic acid sequences and a loop segment, wherein the two nucleic acid sequences that form the double-stranded stem segment have sufficient complementarity to one another to form a double-stranded stem hybrid and are linked and separated by a single-stranded nucleotide segment that forms the loop.

As used herein, the terms “hairpin tip” or “hairpin loop” are used interchangeably and refer to the single stranded loop of the hairpin structure. The phosphodiester bond at the beginning of the hairpin tip is designated 0, with phosphodiester bonds 3′ to the tip numbered +1, +2, etc., and phosphodiester bonds 5′ to the tip numbered −1, −2, etc.

The ability of the Artemis:DNA-PK_{cs} complex to endonucleolytically cleave nucleotides in a site-specific and structure-specific manner allows for the development of assays to analyze nucleic acids suspected of containing 5′ or 3′ overhangs or hairpin motifs. Such assays may be useful for the diagnosis of infectious diseases caused by viruses, bacteria, fungi, inherited mutations, or acquired mutations such as tumors.

For example, this invention provides a method of analyzing a nucleic acid suspected of containing a hairpin motif, comprising:
(a) providing a composition comprising an Artemis:DNA-PKcs complex;
(b) contacting said complex with said nucleic acid under conditions that allow said complex to cleave and open nucleic acid hairpins; and
(c) analyzing said nucleic acid by gel electrophoresis or any of a variety of substituted methods such as fluorescence or radioactivity-based methods.

This invention further provides a method of analyzing a nucleic acid suspected of containing a 3′ or 5′ overhang, comprising:
(a) providing a composition comprising an Artemis:DNA-PKcs complex;
(b) contacting said complex with said nucleic acid under conditions that allow said complex to endonucleolytically cleave said overhang; and
(c) analyzing said nucleic acid by gel electrophoresis or any of a variety of substituted methods such as fluorescence or radioactivity-based methods.

For general NHEJ, the overhang endonucleolytic activity of Artemis is more relevant than hairpin opening Apparently, this aspect of DNA end processing is sufficiently important that cells deficient for it are X-ray sensitive. This activity is insensitive to the 2′-OH of the sugar (because RNA is also cleaved) and largely insensitive to the identity of the base; hence, it is a general structure-specific overhang nuclease.

In studies described below with the D_{FL16.1} and J_{HI} coding end hairpins as free and RAG-generated hairpins (FIGS. 5A, 5B, 6, and 8, and data not shown), the pattern of hairpin opening corresponds to that of opened hairpins generated in the chromosomes of primary thymic T cells and in lymphoid cell lines as determined by Schlissel (Schlissel, 1998). Specifically, the results presented herein confirm the preferential (but not exclusive) hairpin opening 3′ to the hairpin tip. This correspondence suggests that the Artemis:DNA-PK_{cs} hairpin opening activity in vitro functions very similarly to the hairpin opening activity observed in vivo.

When hairpins were opened at positions other than the precise tip, an inverted repeat was generated at the resulting overhang. Such inverted repeats were described initially in V(D)J recombination coding joints in chickens (McCormack, 1989), and were named P (palindromic) nucleotides. They were subsequently identified in V(D)J recombination junctions in all vertebrates. P nucleotides were speculated to arise as a result of opening of hairpin intermediates at non-tip positions (Lieber, 1991). This origin of P nucleotides was firmly established by the identification of hairpin intermediates in DNA-PK_{cs}-deficient and, subsequently, Ku-deficient cells (Both et al., 1992; Zhu et al., 1996). The preferential cleavage of DNA hairpins by the Artemis:DNA-PK_{cs} complex provides an enzymatic basis for completing the understanding of P nucleotide formation.

Junctional diversification at coding joints in V(D)J recombination consists not only of P nucleotide formation, but also nucleotide loss and TdT-dependent additions (Gauss and Lieber, 1996; Lewis, 1994; Lieber, 1991). In fact, most V(D)J recombination junctions do not show any P nucleotides at their coding joints, but rather show nucleotide loss from both coding ends (Gellert, 1997; Lewis, 1994; Lieber, 1998). This may be the result of the endonucleolytic cleavage activity of the Artemis:DNA-PK_{cs} complex. Thus, the Artemis:DNA-PK_{cs} complex may directly participate in the functional diversification.

A role for Ku in the overhang processing or in the hairpin opening by the Artemis:DNA-PK_{cs} complex was not detected. Since Ku improves the affinity of DNA-PK_{cs} by 100-fold (West et al., 1998), one might have expected it to improve the association of the Artemis:DNA-PK_{cs} complex with the target DNA. Potential reasons for the lack of an effect could be as follows. Oligonucleotide substrates have terminal dsDNA ends that may recruit DNA-PK_{cs} efficiently, even in the absence of Ku (Hammarsten and Chu, 1998; Yaneva et al., 1997). This may permit the DNA-PK_{cs} to be stimulated by the open end of the hairpin and the excess 35 bp DNA (in trans) and thereby activate the hairpin opening activity of Artemis. In contrast, RAG-generated hairpins in vivo may require the tight binding affinity and abundance of Ku to help localize DNA-PK_{cs} and hence, the Artemis:DNA-PK_{cs} complex, to the hairpin ends (in cis). In addition, short DNA targets may not permit sufficient space for co-localization of DNA-PK_{cs} and Ku under the tested conditions (Ma and Lieber, 2001; West et al., 1998).

Artemis is a natural enzyme in every vertebrate cell, including humans. As a result of the discovery herein that Artemis functions as a key component of a major DNA repair pathway, this invention further contemplates the identification and development of therapeutic compounds that inhibit Artemis, such as compounds for the treatment of cancers.

Thus, in accordance with another aspect of the present invention, this invention further provides a method for identifying a compound capable of inhibiting Artemis protein activity, wherein the method comprises:
(a) combining the Artemis protein with or without DNA-PK_{cs} and with at least one test compound under conditions permissive for the activity of Artemis;
(b) assessing the activity of Artemis with or without DNA-PK_{cs} and in the presence of the test compound; and
(c) comparing the activity of Artemis with or without DNA-PK_{cs} and in the presence of the test compound with the activity of Artemis with or without DNA-PK_{cs} and in the absence of the test compound, wherein a decrease in the activity of Artemis in the presence of the test compound is indicative of a compound that acts as an inhibitor of Artemis.

“Inhibition” as used herein includes both reduction and elimination of the exonuclease activity of Artemis alone or the endonucleolytic activity of the Artemis:DNA-PK_{cs} complex. Accordingly, a “compound that inhibits Artemis protein activity” refers to a compound that decreases the amount or the duration of the effect of the nuclease activity of Artemis or eliminates Artemis nuclease activity. Such compounds are referred to herein as “Artemis inhibitors.” Inhibitors may include, but are not limited to, proteins, nucleic acids, carbohydrates, antibodies, or any other molecules that decrease or inhibit Artemis activity. “Nuclease activity” as used herein refers to the exonucleolytic activity of Artemis alone or the endonucleolytic activity of Artemis in the Artemis:DNA-PK_{cs} complex.

For example, it was discovered the Artemis protein structure contains a structural fold called the beta-lactamase fold that is necessary for its function, since proteins that are mutants in this domain are inactive. This fold is the same structural fold that is present in the protein beta-lactamase that confers penicillin resistance upon penicillin-resistant bacteria. Thousands of small molecule drugs have already generated to inhibit beta-lactamase, and it is likely that many of these drugs will also inhibit Artemis.

Accordingly, one embodiment of a method for identifying a compound that inhibits the activity of Artemis comprises providing contacting a compound known to inhibit beta-lactamase with Artemis protein, and determining if Artemis activity is inhibited. Thus, in on embodiment the test compounds are selected from beta-lactamase inhibitors. Examples of known beta-lactamase inhibitors include, but are not limited to, clavulanic acid, aztronam, (boric acid, phenylboronic acid (2FDB) and m-aminophenylboronate (MAPS) (Kiener and Waley, Biochem. J., 169, 197-204 (1978); twelve substituted phenylborinic acids, including 2-formylphenylboronate (2FORMB), 4-formylphenylboronate (4FORMB), and 4-methylphenylboronate (4MEPB) (Beesley et al., Biochem. J., 209, 229-233 (1983)); tetraphenylboronic acid (Amicosante et al., J. Chemotherapy, 1, 394-398 (1989)); m-(dansylamidophenyl)-boronic acid (NSULFB) (Dryjanski and Pratt, Biochemistry, 34, 3561-3568 (1995)); and (1R)-1-acetamido-2-(3-carboxyphenyl)ethane boronic acid (Strynadka et al., Nat, Struc. Biol., 3, 688-695 (1996)).

In one embodiment, a compound capable of inhibiting Artemis protein activity identified according to a method of this invention may be used for treating cancer or neoplasms, since rapidly growing tumor cells will not be as prolific if Artemis is inhibited. For example, it has been shown herein that Artemis is needed to open key DNA structures, i.e., hairpins, that are found in lymphoid cells that are actively carrying out V(D)J recombination. Normal lymphoid cells are less sensitive to this inhibition because they only transiently carry out V(D)J recombination. Accordingly, this method provides a method of identifying compounds effective in the treatment of acute lymphoblastic leukemia. Other cancers or neoplasms that can be treated by compounds identified according to the method of this invention include, but are not limited to, leukemia, non-small-cell lung cancer, colon, CNS, melanoma, ovarian, renal, prostate, breast, uterine, liver, and pancreatic cancers, sarcomas of all types, and adenocarcinomas of all types.

Furthermore, such compounds may be also used to treat conditions other than cancer that are also caused by abnormal or altered amounts of Artemis, including but not limited to, proliferative diseases such as polycythemia vera and other conditions including, but not limited to myeloproliferative disorders.

Thus, another aspect of this invention provides a method of ameliorating a condition caused by the activity of Artemis in a patient, comprising administering to the patient an Artemis inhibitor in an amount effective to inhibit Artemis. Such compounds may be useful, for example, in treating cancers such as acute lymphoblastic leukemia based on the role of Artemis in opening hairpins in lymphoid cells.

It is known that the NHEJ pathway is a critical step in the repair pathway of cancerous cells. Based on the discovery herein of the role of Artemis in the NHEJ pathway, it is believed that if the activity of Artemis in cancer cells can be inhibited, the cancer cells will not be able to repair themselves and therefore will be more susceptible to destruction by traditional cancer therapies such as radiation. Therefore, compounds that inhibit the activity of Artemis may be useful in treating conditions caused by the activity of Artemis or by altered or abnormal levels of Artemis.

Accordingly, a further aspect of this invention comprises of enhancing cancer therapy, comprising delivering an Artemis inhibitor to cancerous cells in said patient in an amount effective to inhibit Artemis, followed by administration of a traditional cancer therapy to said patient.

This invention further provides a method of diagnosing a disease or condition in a patient associated with an altered or abnormal amount of Artemis, wherein the method comprises providing a fluid or tissue sample from said patient, and measuring the level of Artemis in the sample. In one embodiment, the assays for Artemis include methods that utilize an antibody that specifically binds Artemis and a label to detect Artemis in human body fluids or in extracts of cells or tissues. The level of Artemis in the sample is then measured by contacting the sample with an antibody that specifically binds Artemis, wherein said antibody is bound to a substrate, and detecting the amount of Artemis that binds to the antibody. Methods of producing antibodies useful for diagnostic purposes may be prepared according to methods known to those skilled in the art. The antibodies may be used with or without modification, and may be labeled by covalent or non-covalent joining with a reporter molecule. A wide variety of reporter molecules, several of which are described above, are known in the art and may be used. Many other methods of measuring the level of a protein are well known to those skilled in the art, and such methods are also included in the scope of this invention.

In order to provide a basis for the diagnosis of a disorder associated with abnormal or altered levels of expression of Artemis, a normal or standard profile for expression is established. This may be accomplished by combining body fluids or cell extracts taken from normal subjects, either animal or human, with a labeled antibody that specifically binds Artemis. The level of binding may be quantified by comparing the values obtained from normal subjects with values from an experiment in which a known amount of Artemis protein is used. Standard values obtained from normal samples may be compared with values obtained from samples from patients who are symptomatic for a disorder. Deviation from standard values is used to establish the presence of a disorder.

With respect to cancer, the presence of a relatively high amount of Artemis in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Artemis protein used in the methods and assays of this invention may be purified from bacterial sources or, preferably, are produced by recombinant DNA techniques, since the gene coding for Artemis is known. Accordingly, this invention also provides a method of producing recombinant Artemis. In one embodiment, the method produces a fusion protein comprising Artemis linked directly or indirectly to an affinity tag. The presence of the affinity tag is useful, for example for purifying Artemis as described below. In one embodiment, the fusion protein is designed so that the tag can be easily cleaved from the protein when desired.

In order to express a biologically active Artemis, the nucleotide sequences encoding Artemis or derivatives thereof may be inserted into appropriate expression vector, i.e., a vector which contains the necessary elements for the transcription and translation of the inserted coding sequence. Methods which are well known to those skilled in the art may be used to construct expression vectors containing sequences encoding Artemis and appropriate transcriptional and translational control elements. These methods include in vitro recombinant DNA techniques, synthetic techniques, and in vivo genetic recombination. Such techniques are described in Sambrook, J. et al. (1989; Molecular Cloning. A Laboratory Manual, ch. 4, 8, and 16-17, Cold Spring Harbor Press, Plainview, N.Y.); and Ausubel, F. M. et al. (1995 and periodic supplements; Current Protocols in Molecular Biology, ch. 9, 13, and 16, John Wiley & Sons, New York, N.Y.).

For example, one embodiment of the present invention is a method to produce an isolated Aremtis protein comprising the steps of (a) culturing a recombinant cell comprising a nucleic acid molecule encoding a protein of the present invention to produce the protein and (b) recovering the protein therefrom. The phrase “recovering the protein” refers simply to collecting the whole fermentation medium containing the protein and need not imply additional steps of separation or purification. Artemis protein of the present invention can be purified using a variety of standard protein purification techniques, such as described below.

A variety of expression vector/host systems may be utilized to contain and express sequences encoding Artemis. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The invention is not limited by the host cell employed.

The Artemis protein and fusion proteins are expressed in the respective expression systems under the control of a suitable promoter. In case of the expression in eukaryotes, all known promoters, such as SV40, CMV, RSV, HSV, EBV, beta-actin, hGH or inducible promoters, such as, e.g. hsp or metallothionein promoters are suitable therefore.

In another embodiment, Artemis is expressed as a fusion protein comprising Artemis linked directly or indirectly to an affinity tag. Accordingly, one embodiment of this invention provides a method of producing a fusion protein, comprising (a) providing an expression vector comprising a nucleic acid sequence that encodes an affinity tag; (b) inserting a polynucleotide that encodes Artemis into the vector in a manner that allows the polynucleotide to be operatively linked to the vector; (c) transfecting cells with said vector under conditions that allow expression of Artemis and the affinity tag to produce a fusion protein comprising Artemis linked to the affinity tag.

Tagging is a powerful and versatile strategy for detecting and purifying proteins expressed by cloned genes. To utilize this feature, protein expression vectors are typically engineered with a nucleotide sequence that encodes the affinity tag. For example, the Artemis gene is cloned in-frame relative to the tag and, upon expression, the Artemis protein is synthesized as a fusion protein with the peptide tag. Fusion protein detection and/or purification is mediated by binding partners to the tag.

The term “affinity tag” is used herein to denote a polypeptide segment that can be attached to a second polypeptide such as Artemis protein to provide for purification of the second polypeptide or provide sites for attachment of the second polypeptide to a substrate. In principal, any peptide or protein for which an antibody or other specific binding agent is available can be used as an affinity tag. Affinity tags include, but are not limited to a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952-4 (1985)), substance P, Flag™ peptide (Hopp et al, Biotechnology 6:1204-1210 (1988)), streptavidin binding peptide, maltose binding protein (Guan et al., Gene 67:21-30 (1987)), cellulose binding protein, thioredoxin, ubiquitin, T7 polymerase, or other antigenic epitope or binding domain. See, in general, Ford et al., Protein Expression and Purification 2:95-107 (1991). Other examples of commonly used affinity tags include c-myc, beta-galactosidase, avidin, maltose binding protein (MBP), influenza A virus, and haemagglutinin. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

In one embodiment, the fusion proteins according to the invention comprise Artemis linked directly or indirectly to an affinity tag, such as a heterologous protein, polypeptide or a functionally active peptide. According to the invention, the affinity tag is selected such that it has a high affinity or a specific binding property for a binding partner (e.g., antibodies) that is coupled to a solid carrier. According to the invention, the adsorption of the fusion protein to the solid carrier may be effected e.g. by covalent binding or via affinity. To facilitate heterologous membrane protein purification (through isolation of the heterologous membrane protein from other ICM components), an affinity tag is engineered into the protein-coding sequence.

In another embodiment, a fusion protein of this invention comprises an Artemis:DNA-PK_{cs} protein. This fusion protein may also comprise an affinity tag as described above.

According to the invention, the fusion of Artemis to a peptide tag is to be effected such that the enzymatic function of Artemis is not negatively affected. According to a particular aspect of the present invention, a short peptide spacer is inserted between the Artemis sequence and the peptide tag sequence.

In one embodiment, the peptide tag may form a covalent bond with, or has a high affinity to, a binding partner for the tag that is coupled to a solid carrier. Examples of such binding partners include, but are not limited to, heavy metal ions or specific anti-peptide tag antibodies

According to a particular aspect of the present invention, the fusion protein is immobilized by being bound to the solid carrier. According to the present invention, the solid carrier may be provided as matrix. Natural and synthetic matrices, such as sepharose, agarose, gelatin, acrylate etc. may be used as the matrix to which the affinity carrier adsorbs.

Accordingly, another embodiment of this invention provides a method of purifying Artemis, comprising expressing Artemis as a recombinant protein with an affinity tag, contacting the protein with a matrix comprising a binding partner for the tag, washing the matrix with an eluent that removes extraneous materials but does not remove the protein, and releasing the protein from the matrix. This method allows purified Artemis protein to be generated hundreds of times more easily than purification of native protein, and has important relevance for the use of Artemis as a drug target.

Alternatively, the Artemis protein itself may be produced using chemical methods to synthesize the amino acid sequence of Artemis, or a fragment thereof. For example, peptide synthesis can be performed using various solid-phase techniques (Roberge, J. Y. et al. (1995) Science 269:202-204) and automated synthesis may be achieved using a synthesizer (Perkin Elmer).

As discussed above, the spectrum of activities of Artemis is shifted from exonucleolytic to endonucleolytic upon complex formation with and phosphorylation by DNA-PK_{cs}. There are other examples of nucleases that have both exonuclease and endonuclease activity. E. coli RecBCD, although an endonuclease, acts exonucleolytically while translocating on its DNA substrate as a helicase (Kowalezykowski and Eggleston, 1994). In eukaryotes, FEN-1 has also been shown to have both exonuclease and endonuclease activity. Relevant to the Artemis mutant described here, it has been reported that some mutations in FEN-1 affect the exonucleolytic activity but not the endonucleolytic activity and vice versa (Xie et al., 2001), despite the fact that there is only one nucleolytic active site in FEN-1 (Lieber, 1997; Shen et al., 1996).

Although Ku70, Ku86, DNA ligase IV, and XRCC4 exist in all eukaryotes, including yeast and plants (Lieber, 1999; West et al., 2000), DNA-PK_{cs} and Artemis are thus far only detectable in vertebrates (Moshous et al., 2001). Clearly, the use of a transpositional excision mechanism that generates hairpins, namely, V(D)J recombination, is one obvious distinction of vertebrates.

Based on this, there appears to be no need for a hairpin opening activity in the absence of hairpin or hairpin-like DNA structures in non-vertebrate eukaryotes. Mre11, together with Rad50 and Xrs2, have been proposed as candidates for opening hairpins in vivo (Paull and Gellert, 1998). However, the biochemical support for such an in vivo role of Mre11 is compromised by the fact that no hairpin opening has been demonstrated under physiologic divalent salt conditions (Paul, 1999; Paull and Gellert, 1998; Paull and Gellert, 1999). Rather, Mre11 opening of hairpins has only been achieved in manganese buffers, which can distort the physiologic spectrum of nuclease activities. Moreover, Nbs1 (Xrs2) mutant patients and cells from them appear to have normal V(D)J recombination (Harfst et al., 2000; Yeo et al., 2000). In marked contrast, patients with two mutant Artemis alleles can not form coding joints, and thus have no mature B or T cells; this indicates that Mre11 is unable to provide any backup function for hairpin opening in vertebrates when Artemis is absent (Moshous et al., 2001; Moshous et al., 2000). In yeast, where homologues of Artemis and DNA-PK_{cs} do not appear to exist, artificial cruciforms require the Rad50/Xrs2/Mre1 1 complex for resolution (Lobachev et al., 2002). Though cruciforms have hairpins within their structure, it is not clear from that study that Mre11 is actually cleaving those hairpins, or whether the Rad50/Xrs2/Mre1 1 complex plays another role in the resolution of such structures.

The role of the Artemis:DNA-PK_{cs} complex as an overhang nuclease in NHEJ may be served by other proteins (such as FEN-1 (Rad27 in S. cerevisiae)) (Wu et al., 1999). Due to the structural resemblance of overhangs to hairpin structures (FIG. 9), the evolution of the Artemis:DNA-PK_{cs} complex may have made other overhang nucleases in NHEJ unnecessary in vertebrates. The radiation sensitivity of Artemis and of DNA-PK_{cs} mutants (Hendrickson et al., 1991; Nicolas et al., 1998) suggests the 5′ and 3′ overhang processing by this complex cannot be accomplished by any of the other nucleases in the cell.

The invention may be better understood with reference to the accompanying examples that are intended for purposes of illustration only and should not be construed as, in any sense, limiting the scope of the present invention, as defined in the claims appended hereto. While the described procedures in the following examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLE 1

### Construction of GST-Artemis and Artemis-myc-His Expressing Plasmids

Full-length human Artemis cDNA was amplified by recombinant Pfu DNA polymerase (Stratagene, Cat. No. 600154) using a Human Thymus Matchmaker cDNA Library (Clontech, Cat. No. HL4057AH) as the template. Fragment ARTI was amplified using primers BamH1ARTcDNA5′ (5′-CGGGATCCATGAOTTCTTTCGAGGG-3′) and Not1ARTcDNA3′ (5′ ATAAGAATGCGGCCGCTTAGGTATCTAAGAG-3′). Fragment ART2 was amplified using primers Kpn1ARTcDNA5′N (5′-GGGGTACCGCTATGAGTTCTTTCGAGGG-3′) and Not1ARTcDNA3′w/oSTOP (5′-ATAAGAATGCGGCCGCCAGGTATCT-AAGAGTGAGC-3′). A GST-Artemis expressing plasmid was constructed by cloning fragment ART1 into the pEBG vector after BamHI/NotI-digest. An Artemis-myc-His expressing plasmid was generated by ligating fragment ART2 into the pcDNA6/myc-His vector (Version A, Invitrogen, Cat. No. V221-20) after KpnI/Not1-digest. The integrity of the inserts was checked by sequencing. The point mutant Artemis (D165) was generated with the QuickChange SiteDirected Mutagenesis kit (Stratagene, Cat. No. 200516). Primers used for mutagenesis were Di65N/F 5′-CAAAGTGTATATTTGAATACTACGTTCTGTG-3′ and D165N/R 5′-CACAGAACGTAGTATTCAAATATACACTTTG-3′. Subsequently, the D165N cDNA ORF was confirmed by sequencing.

### EXAMPLE 2

### Protein Purification

GST-Artemis and Artemis-myc-His expressing plasmids pEBG-huArtemis, pcDNA6/huArtemis-myc-His, pcDNA6/ARM19 (for the expression of D165N mutant) were transfected into 293T cells by calcium phosphate precipitation (Wigler et al., 1979). For the purification of GST-Artemis, cells were collected, washed once with 1×PBS and resuspended in buffer A (25 mM Tris, pH 8.0, 500 mM KCl, 0.5 mM EDTA, 10% glycerol, 1 mM DTT, 0.05% Triton X-100) with protease inhibitors (0.1 mM phenylmethylsulfonylfluoride (PMSF), 1 μg/ml Leupeptin, and 1 μg/ml Pepstatin A). Then the cell suspension was sonicated and centrifuged at 24,500 g for 30 minutes at 4° C. The supernatant was mixed with Glutathione (GSH)-agarose (Sigma, St. Louis, Mo.) and incubated overnight at 4° C. After washing the beads, proteins were eluted with buffer C (50 mM Tris, pH 8.0, 150 mM KCl, 20% glycerol, 1 mM DTT, 10 mM GSH). Eluted protein fractions were dialyzed against buffer D (20 mM Tris, pH 7.5, 100 mM KCl, 20% glycerol, 0.5 mM PMSF, 5 mM DTT) and frozen in aliquots at −80° C.

For the purification of Artemis-myc-His, transfected cells were collected, washed in 1×PBS, and resuspended in buffer E (50 mM Na₂PO₄, pH 8.0, 500 mM NaCl, 20 mM 13-mercaptoethanol (β-ME), 0.1% Triton X-100) and 20 mM imidazole (designated as buffer E-20) with protease inhibitors (as described above). Then the cell suspension was sonicated and centrifuged as above and the supernatant was mixed with Ni-nitrilotriacetic acid (Ni-NTA) agarose (Qiagen, Valencia, Calif.) and incubated for 1 hour to overnight. Washing was performed in buffer E-20, E-30, E-40, and E-50 (step washes with increasing concentrations of imidazole). Artemis-myc-His appeared in the flow-through and was completely eluted by the step of 40 mM imidazole wash. The fractions containing Artemis-myc-His were then pooled together, mixed with anti-myc antibody (clone 1-9), and then protein G Sepharose (Amersham Pharmacia Biotech, Piscataway, N.J.). The antibody-protein G Sepharose beads were incubated with protein fractions overnight and washed thoroughly with buffer F (25 mM HEPES, pH 7.9, 650 mM KCl, 10 mM MgCl₂, 0.1% NP-40). The Artemis bound protein G beads were finally washed with buffer G (25 mM HEPES, pH 7.9, 10 mM MgCl₂, 2 mM TT) and frozen at −80° C. These immunobeads were then used as the Artemis enzyme.

The concentration of purified proteins was estimated by comparing to bovine serum albumin standards on a Coomassie blue stained SDS-PAGE gel. The identity of Artemis was determined using Western blots probed with anti-GST (BD PharMingen, San Diego, Calif.) and anti-myc antibodies.

Native DNA-PK_{cs} was purified as described (Chan et al., 1996) except that HeLa cells were used as the source for purification. C-terminal His tagged Ku70 and non-tagged Ku 86 were co-expressed in the baculovirus system and purified as described (Yaneva et al., 1997). GST tagged core RAG-1 (a.a. 384 to 1008) and GST tagged RAG-2 (a.a. 1 to 383) were co-expressed and purified on GSH-agarose (Cortes et al., 1996; Sawchuk et al., 1997). C-terminal truncated HMG1 was expressed in bacteria and purified on Ni-NTA column (West and Lieber, 1998). C-terminal His tagged DNA ligase IV and non-tagged XRCC4 were co-expressed in a baculovirus system and purified as previously described (NickMcElhinny et al., 2000).

### EXAMPLE 3

### Oligonucleotides

The oligonucleotides used in this study were synthesized by Operon Technologies (Alameda, Calif.) or the Microchemical Core Facility (Morris Cancer Center, USC). Oligonucleotides. The sequences of the oligonucleotides are as follows. In FIG. 3, substrate (dA)₂₀ has a sequence of 20 dAs and (dT)₂₀ (YM-145) has a sequence of 20 dT's. In FIG. 4A, the substrate was composed of YM-130 (5′-TTTTTTTTTTTTTTACTGAGTCC TACAGAAGGAT-3′) and YM-68 (5′-GATCCTTCTGTAGGACTCAGT-3′). In FIG. 4B, the substrate was composed of YM-149 (5′-ACTGAGTCCTACAGAAGGATCTTTTTT TTTTTT-3′) and YM-68. YM117 (5′-GATTACTACGGTAGTAGCTACGTAGCTCTACCG TAGTAAT-3′, sequence without the 5′ G is a hairpin of marine D_{FL16.1} coding end sequence) was used for FIGS. 5A, 5B, and 6. YM-105 (5′-CGACTGCGTCTAGACAGCTCACCCGGCCGGGTGAGCTGTCTAGACG-3′) was used for FIG. 7. In FIG. 8, the 12-RSS containing oligonucleotides were composed of KY28 and KY29, and the 23-RSS containing oligonucleotides were composed of KY36 and KY37 (Yu and Lieber, 2000). The exogenous 35 bp DNA used as a DNA-PK_{cs} cofactor in FIGS. 4 to 8 was the same as described (West et al., 1998). The sequence of the 35 bp DNA used for FIG. 10 was the same as described (West et al., 1998). In FIG. 11, the substrate with GC-rich end (2% cutting efficiency) was composed of YM-107 (labeled strand, 5′-CGGCCGTACAOTCTGATCGCTCAT-3′) and YM-108 (5′-GATGAGCGATCAGACTGTACGGCCG-3′); the other substrates have the same sequences as YM-107/YM-108 except the shown 6 by at the labeled end.

### EXAMPLE 4

### In Vitro Immunobead Pull-Down Assay

20 μL of protein G Sepharose was mixed with a total of 15 pmol of monoclonal anti-DNA-PK_{cs} antibodies (clones 42-27, 25-4, and 18-2) or 15 pmol of monoclonal anti-Ku antibodies (clones 111 and N3H10 (Neomarkers, Fremont, Calif.)) in 20 mM HEPES, pH 7.4, 10 mM MgCl₂, 10% glycerol, 2 mM DTT, 0.1 mg/ml BSA and different concentrations of KCl (0 mM, 100 mM, or 500 mM). 2.5 pmol of DNA-PK_{cs} and 2.5 pmol of Ku were added to anti-DNA-PK_{cs} immunobeads and anti-Ku immunobeads, respectively. Then 1.8 pmol of GST-Artemis was mixed in. The reactions (final volume=50 μL) were incubated at 4° C. for 1.5 hours. The immunobeads were then washed with 1 ml of the corresponding binding buffers for 3 times and analyzed by Western blotting.

To perform the assay (FIGS. 2A and 2B), purified DNA-PK_{cs} and GST-Artemis were loaded in lanes 1 and 2, respectively. DNA-PK_{cs} (lanes 5 to 7) or Ku (lanes 10 to 12) were immobilized on antibody protein 0 Sepharose beads at different concentrations of KCl. As a control, Anti-myc antibody was used in lanes 3 and 8. DNA-PK_{cs} and Ku were excluded from lanes 4 and 9, respectively. After GST-Artemis was added, the beads were washed with the corresponding binding buffer, then analyzed by Western blotting with anti-DNA-PK_{cs} antibody (portion above the dotted line) and anti-GST antibody (portion below the dotted line). GST-Artemis has an apparent molecular weight of 120 kD on this 8% SDS-polyacrylamide gel, and the bands of smaller sizes in lane 2 represent C-terminal degradation products of GST-Artemis. Positions of GST-Artemis, immunoglobulin heavy chain and light chain are indicated on the right. Protein molecular weight standards (in kDa) are indicated on the left. The transferred membrane was cut at approximately the position of the 150 kD marker; the top portion was probed with anti-DNA-PK_{cs} antibodies, and the bottom portion was probed with anti-GST antibody.

To confirm that Ku was indeed immobilized on the beads, the bottom portion of the membrane was stripped and reprobed with anti-Ku antibodies (D6D8, D6D9, 2D9, and anti-Ku70 (Yaneva et al., 1997)). FIG. 2(B) shows the Coomassie staining of immunoprecipitation samples with anti-myc antibody is shown in the upper panel. Purified DNA-PK_{cs} was loaded in lane 1. Cell lysates were subjected to immunoprecipitation with anti-myc antibody and the immunobeads were loaded in lane 2 (from transfection with empty vector) and lane 3 (from transfection with Artemis-myc-His expressing vector), respectively. Protein molecular weight standards are indicated on the left. Positions of DNA-PK_{cs}, Artemis-myc-His, immunoglobulin heavy chain and light chain are indicated on the right. Samples used for the top panel were also subject to Western blotting analysis and the result is shown in the lower panel. The blot was probed with monoclonal anti-DNA-PK_{cs} antibodies (42-27, 25-4, and 18-2).

### EXAMPLE 5

### Immunoprecipitation of DNA-PK_{cs} from Artemis Transfected Cells

293T cells transfected with empty vector or Artemis-myc-His expressing plasmid were harvested, washed in 1×PBS, and then resuspended in 25 mM HEPES, pH 7.4, 150 mM KCl, 10 MM MgCl₂, 10% glycerol, and 2 mM DTT supplemented with protease inhibitors (as described above). Cells were lysed by sonication and centrifuged as above. Anti-myc antibody and protein G Sepharose were added to the cell lysates and binding was allowed to proceed for 12 to 16 hrs. After being washed extensively in the same buffer, the immunobeads were denatured in sample loading buffer and fractionated on an 8% SDS-PAGE then either stained with Coomassie blue or analyzed by Western blotting with anti-DNA-PISS antibodies.

### EXAMPLE 6

### In Vitro Nuclease Assays

Nuclease assays without RAGs were carried out in a total volume of 10 μL with a buffer composition of 25 mM Tris, pH 8.0, 10-50 mM NaCl or KCl, 10 mM MgCl₂, 1 mM DTT, and 50 ng/μL of BSA unless otherwise specified. To the buffer mixture, Artemis was added to 2.75 pmol, and DNA-PK_{cs} and Ku were added to 1.25 pmol each. 0.25 mM of ATP (or ADP, ATP-y-S, AMP-PNP) and 0.5 PM of 35 bp DNA were included where DNA-PK_{cs} was used. Reactions were incubated at 37° C. for 30 minutes. In reactions including DNA-PK_{cs} inhibitors, reaction mixtures without the substrate were incubated on ice for 15 minutes before the addition of the substrate and the subsequent incubation at 37° C. In FIG. 7, pre-phosphorylation of Artemis-myc-His immunobeads was carried out under DNA-PK kinase assay conditions. After washing the treated immunobeads with buffer F for three times and the nuclease assay buffer for two times, the beads were used for the nuclease reactions. In the hairpin opening of RAG-generated hairpins (FIG. 8), the reactions contained 25 mM K-HEPES, pH 7.4, 50 mM KCl, 10 mM MgCl₂, 1 mM DTT, 0.25 pmol of labeled 12-RSS double-stranded oligonucleotides (KY28/KY29) and an equal amount of unlabeled 23-RSS double-stranded oligonucleotides (KY36/KY37), 1 pmol of RAGs (assuming that the RAG complex consists of two RAG-1 and two RAG-2 subunits), 2 pmol of HMG1, 2.75 pmol of Artemis-myc-His, and 1.25 pmol of DNA-PK_{cs} (with ATP and 35 bp DNA as described above). For the sequential reactions, substrates were incubated with RAG complex alone first at 37° C. for 60 minutes, extracted with or without phenol/chloroform, then Artemis-myc-His and DNA-PK_{cs} were added, followed by another 30-minute incubation at 37° C. Reactions with the RAG complex, Artemis, and DNA-PK_{cs} added simultaneously were incubated for 90 minutes at the same temperature. After incubation, reactions were stopped by adding an equal volume of formamide gel loading buffer and beating at 100° C. for 5 minutes. DNA was resolved on 12% denaturing polyacrylamide gels. The gels were then dried and exposed to a PhosphorImager screen. Data was analyzed by ImageQuant software (v5.0).

### EXAMPLE 7

### DNA-PK Kinase Assay

The DNA-PK_{cs} kinase assay was performed in a total volume of 20 μL which contains 10 mM Tris (pH 7.5), 1 mM EDTA, 10 mM MgCl₂, and 1 mM DTT, 0.3 μM 35 bp DNA (YM-8/YM-9), and 165 nM of [α-³²P]ATP (3000 Ci/mmol, PerkinElmer). DNA-PKcs was added to 60 nM to a final concentration of 60 nM and GST-Artemis and DNA ligase IV/XRCC4 (assume the complex of DNA ligase IV/XRCC4 contains one DNA ligase IV and two XRCC4 subunits) were added to 180 nM and 50 nM, respectively. Reaction mixtures were incubated at 37° C. for 30 minutes and fractionated on an 8% SDS-PAGE. The gel was dried and then exposed to a PhosphorImager screen, and the image was obtained by using PhosphorImager 445SI (Molecular Dynamics, Sunnyvale, Calif.) and analyzed with ImageQuant software (v5.0).

### EXAMPLE 8

### Artemis and DNA-PK_{cs} Form a Stable Complex In Vitro that is Independent of DNA Ends

Cells from patients with mutations in the Artemis gene have been shown previously to be defective for V(D)J recombination in a manner that is indistinguishable from cells defective for DNA-PK_{cs} (Moshous et al., 2001; Moshous et al., 2000). Therefore, it was hypothesized that the Artemis protein and DNA-PK_{cs} might be part of a larger complex and involved in similar steps in V(D)J recombination.

To test this hypothesis, human cDNA of Artemis was cloned into either GST N-terminal or myc-his C-terminal fusion protein vectors (see Experimental Procedures). Interactions between Artemis and DNA-PK_{cs} and between Artemis and Ku were first tested in vitro using immunobead pull-down experiments. DNA-PK_{cs} was immobilized on protein G Sepharose beads using monoclonal antibodies against DNA-PK_{cs}, and then purified GST-Artemis was added. After incubation, the beads were washed stringently to remove any unbound molecules, and the pull-down fraction was analyzed by Western blotting.

As shown in FIG. 2A, it was observed that GST-Artemis associated with DNA-PK_{cs} at 0 and 100 mM KCl (lanes 5 and 6), but the interaction was unstable at 500 mM KCl (lane 7). The top portion of the membrane shows that DNA-PK_{cs} was present on the beads under all salt conditions. (Note that while Coomassie staining of the DNA-PK_{cs} shows that the majority of it is full-length (see FIG. 2B, lane 1), the residual lower molecular weight fragments transfer much more efficiently than the full-length form in Western blotting, thus explaining the apparent presence of prominent degradation products.)

Next, since it was observed that Ku associates with DNA-PK_{cs} on DNA ends to form the DNA-PK holoenzyme, a corresponding experiment was performed using immobilized Ku instead of immobilized DNA-PK_{cs}. As shown by lanes 9-12 in FIG. 2A, there was no evidence of interaction between Artemis and Ku. After probing the Western blot with anti-GST antibodies, the bottom portion of the membrane was stripped and reprobed for Ku, which confirmed that Ku was indeed present on the beads under all salt concentrations (data not shown).

Based on the above results, it was concluded that Artemis and DNA-PK_{cs} form a stable complex in physiologic ionic strength in the absence of DNA in vitro. However Ku and Artemis do not form such a complex. Thus, the Artemis:DNA-PK_{cs} complex does not rely on DNA termini or Ku for stability. Even in the presence of linear dsDNA, interaction between Ku and Artemis was not detected in an electrophoretic mobility shift assay (data not shown).

This raises the possibility that this is the functional state of Artemis inside the cell, given that DNA-PK_{cs} is a relatively abundant nuclear protein and the level of Artemis expression is low (Anderson and Carter, 1996; Moshous et al., 2001). This would be consistent with the phenotypic similarity concerning X-ray sensitivity, as well as signal joint formation but failure of coding joint formation in Artemis and DNA-PK_{cs} mutants.

### EXAMPLE 9

### Artemis and DNA-PK_{cs} Form a Stable Complex In Vivo

To test whether Artemis and the 469 kDa DNA-PK_{cs} form a complex in vivo, Artemis-myc-His expression plasmid was transfected into 293T cells, and then Artemis and any potentially associated protein(s) were immunoprecipitated from transfected cells using anti-myc antibody bound to protein G Sepharose beads. DNA-PK_{cs} was co-immunoprecipitated as identified by size on Coomassie stained gels (FIG. 2B, upper panel, lane 3). The identity of DNA-PK_{cs} was confirmed by Western blotting (FIG. 2B, lower panel). This interaction was stable at 100 mM KCl with or without nonionic detergent, but was unstable at 500 mM KCl (data not shown). The control immunoprecipitation in which the expression vector lacking the Artemis cDNA was transfected showed no detectable DNA-PK_{cs} (FIG. 2B, upper and lower panel, lane 2). These results strongly suggest that Artemis and DNA-PK_{cs} form a stable complex in vivo.

### EXAMPLE 10

### DNA-PK_{cs} Phosphorylates Artemis

A DNA-PK kinase assay was performed to determine whether Artemis is a phosphorylation substrate of DNA-PK_{cs}. The results are shown in FIG. 10. DNA-PK_{cs} was incubated alone (i.e., with no protein substrate; lanes 1 and 2), or with DNA ligase IV/XRCC4 (positive control, lanes 3 and 4) or GST-Artemis (lanes 5 and 6). The low amount of XRCC4 and Artemis phosphorylation in the absence of 35 bp dsDNA is thought to be due to a low level of DNA-PK_{cs} activity that is DNA-independent (Hammarsten et al., 2000; Yaneva et al., 1997). Positions of phosphorylated proteins are indicated on the right. Bands lower than GST-Artemis represent degradation products of GST-Artemis (see also FIG. 2A).

The results of the DNA-PK kinase assay demonstrated that Artemis is indeed a prominent phosphorylation target of DNA-PK_{cs}, as illustrated by the DNA dependent phosphorylation (lanes 5 and 6). Therefore, DNA-PK_{cs} not only forms a physical complex with Artemis, but it is also able to efficiently phosphorylate Artemis upon complex formation. The results further show that this activity is dependent on DNA ends. These results imply that the Artemis. DNA-PK_{cs} nuclease complex would be ideally responsive to pathologic dsDNA breaks.

### EXAMPLE 11

### Artemis is a Single-Strand Specific Nuclease with a 5′ to 3′ Exonucleolytic Polarity

In the original identification of Artemis, the homology of the N-terminal region of Artemis to the SNM1 protein of S. cerevisiae was described (Moshous et al., 2001). The SNM1 protein and the homologous region of Artemis are predicted to contain beta-lactamase folds, which are known to function enzymatically in reactions that utilize water molecules as nucleophiles to break covalent bonds. For this reason, the initial characterizations of Artemis alone according to this invention included testing for nucleolytic activity.

In the nuclease assay, time course experiments were performed using ssDNA labeled at its 5′ end using polynucleotide kinase or at its 3′ end using [α-³²P] dideoxyadenosine triphosphate (ddATP) and terminal deoxynucleotidyl transferase (TdT). The results are shown in FIG. 3. Lanes M1 and M2 contain size standards generated by digesting the top strand of the substrate with Klenow fragment for 30 minutes and 60 minutes, respectively. A time course of the degradation of the substrate by Artemis-myc-His alone (lanes 2 to 4) or Artemis and DNA-PK_{cs} (lanes 5 to 7) is shown. Sizes of the major products are indicated on the right. Diagrams in the right margin show the cleavage positions (shown by arrowheads) in the substrate that result in the corresponding degradation products (the bands pointed by arrows).

As shown in FIG. 3, the 5′-radiolabeled single-stranded (ss) DNA yielded only a 1-nucleotide product (lanes 2 to 4). However, a 3′-radiolabeled ssDNA yielded a ladder of products terminating at 2 nucleotides (lanes 6 to 7), suggesting that nucleic acid targets must be larger than 2 nucleotides for Artemis to bind and/or cleave. The size of the 2-nucleotide product was confirmed by treating the substrate with snake venom phosphodiesterase.

Overall, these results suggest that Artemis alone possesses 5′ to 3′ exonuclease activity on ssDNA. If Artemis were a 3′ exonuclease, then initially the 3′ A would be removed and only a mononucleotide product would be observed (lanes 6 and 7). In addition, the 5′-labeled ssDNA would yield a degradation ladder. The 51 to 3′ single-stranded exonuclease activity of Artemis appears to be processive rather than distributive because there is extensive cleavage of a large fraction of the single-stranded molecules while other molecules in the same population have not been cleaved at all (FIG. 3, lanes 6 and 7, and data not shown).

The specificity of Artemis on ssDNA was examined with dsDNA with GC- or AT-rich ends (FIG. 11A) and with DNA having increasing numbers of terminal mismatches (FIG. 11B). The double-stranded oligonucleotides labeled with T4 polynucleotide kinase (T4 PNK) on one strand (as indicated by the asterisk) were incubated with Artemis-myc-His. T4 PNK catalyzes the transfer of the γ-phosphate of ATP to the 5′-terminus of double-stranded DNA having a 5′-OH. The results indicated that Artemis has very limited nucleolytic activity on dsDNA molecules (FIG. 11A). This indicates that Artemis is relatively specific for ssDNA with no endonuclease activity on dsDNA. The preference of Artemis for GC-rich ends suggested that “breathing” (spontaneous and partial unwinding) at dsDNA ends might account for this. This hypothesis was supported by the fact that the exonucleolytic activity of Artemis increases markedly on substrates with an increasing number of terminal mismatches (FIG. 11B). Altogether, these results indicate that Artemis alone is a 5′ to 3′ single-strand specific exonuclease. It is also noteworthy that the 5′-exonucleolytic activity is strongly dependent on the presence of a 5′ phosphate and is equivalently active on RNA as it is on DNA (data not shown). Temperature and ionic strength dependence studies showed that 37° C. and 50 mM KCl are the optimal conditions for 5′-exonucleolytic activity of Artemis (data not shown). Importantly, Artemis is only active as a nuclease in buffers containing Mg²⁺ and is inactive in corresponding buffers containing Mn²⁺ and Zn²⁺ (data not shown).

### EXAMPLE 12

### DNA-PK_{cs} Regulates the Overhang Endonucleolytic Activity of Artemis

In order to determine how Artemis acts on substrates with long 5′ overhangs, a substrate comprising a double-stranded oligonucleotide with a (dT)₁₅ 5′ overhang or a (dT)₁₅ 3′ overhang end-labeled with T4 PNK on the long strand (as indicated by the asterisk) was digested with Klenow fragment. The results are shown in FIGS. 4A and 4B. A time course of the degradation of the substrate by Artemis-myc-His alone is shown in lanes 2 to 4, and the time course of the degradation of the substrate by Artemis and DNA-PK_{cs} is shown in lanes 5 to 7. A control reaction of DNA-PK_{cs} and the substrate is shown in lane 8 of FIG. 4B. Lanes M1 and M2 contain size standards generated by digesting the top strand of the substrate with Klenow fragment for 30 min and 60 min, respectively.

The time courses of Artemis action on substrates with a (dT)₁₅ 5′ overhang showed that the 5′ mononucleotide was the initial cleavage product, with no intermediate products (FIG. 4A, lanes 1 to 4). Therefore, it appears that Artemis recognizes long 5′ overhangs as ssDNA. With the substrate composed of a 21 bp double-stranded portion and a (dT)₁₅ 3′ overhang (FIG. 4B), the products indicated 5′ exonucleolytic cleavage (lanes 1 to 4, bottom of gel). The 5′ exonucleolytic cleavage occurred 2 nucleotides from the 5′ end on some dsDNA substrates (Figure lanes 2 to 4), instead of 1 nucleotide, as was observed for purely ssDNA (FIG. 3).

The nucleolytic properties of Artemis alone were important to establish; however, the in vivo protein interaction studies described above indicate that Artemis functions as a complex with DNA-PK_{cs}. Therefore, the nuclease activity of Artemis along with DNA-PK_{cs} was evaluated. In the presence of DNA-PK_{cs}, Artemis showed a very significant shift in the ratio of cleavage products on DNA with long 5′ overhangs (FIG. 4A, lanes 2 to 4 versus 5 to 7). With DNA-PK_{cs} present instead of only the 5′ mononucleotide product, Artemis generated a series of endonucleolytic cleavages internal to the 5′ end, but with a significant predilection for cleavage at the position that yields a blunt-ended dsDNA product and a labeled 15 nucleotide ssDNA product (FIG. 4A, lanes 6 and 7). DNA-PK_{cs} alone has no such activity (data not shown), and Artemis alone only generates the 5′ mononucleotide product as described above (FIG. 4A, lanes 2 to 4). Labeling at the 3′ end of the same strand confirmed these findings (data not shown). Interestingly, at shorter times (FIG. 4A, lane 6), predominantly the 5′ mononucleotide and the 15-nucleotide product from the overhang endonucleolytic cleavage reaction were observed.

Long 3′ overhangs were also tested for cleavage by Artemis in the presence of DNA-PK_{cs} using the dsDNA substrate with a (dT)₁₅ 3′ overhang. DNA-PK_{cs} enabled Artemis to cleave the 3′ overhang (FIG. 4B, lanes 1 and 5 to 7). The cleavage products at early times were predominantly in the single-stranded tail 4 to 6 nucleotides from the single-strand to double-strand transition point (FIG. 4B, lane 5). At longer times, the distribution of products ranged from cleavage at the single-strand to double-strand transition point to positions outward along the single-stranded overhang for approximately 10 nucleotides. These results indicate that DNA-PK_{cs} not only forms a complex with Artemis, but also regulates the spectrum of its activities. Because DNA-PK_{cs} binds at the single-strand/double-strand transitions such as found at DNA with 3′ or 5′ overhangs, Artemis would necessarily be recruited to these locations because of its association with DNA-PK_{cs}. This may permit a very low or undetectable overhang cleavage activity to become a relatively strong one.

### EXAMPLE 13

### DNA-PKcs Confers DNA Hairpin Opening Activity on Artemis

Because DNA-PK_{cs} mutants are arrested in V(D)J recombination at the hairpin opening step and because both Artemis and DNA-PK_{cs} mutant mammals have indistinguishable V(D)J recombination phenotypes, it was of interest to determine whether Artemis would act on hairpins. A hairpin with the sequence of the marine D_{FL16.1} coding end was synthesized and tested as a substrate for Artemis. As shown in FIG. 5A, 5′ to 3′ exonucleolytic activity of Artemis alone at the non-hairpin (labeled) end of the hairpin DNA substrate was observed, resulting in the generation of a 2 nucleotide product (lane 2). Though one might have expected a 1 nucleotide product based on the earlier studies (FIG. 3), it appears that the exonucleolytic action of Artemis at DNA ends is somewhat affected by the precise DNA sequence, such that here a 2 nucleotide product results.

No hairpin opening by Artemis alone was detectable (FIG. 5A, lane 2), and addition of Ku did not alter the spectrum of Artemis activities (FIG. 5A, lane 3). However, the addition of DNA-PK_{cs} substantially shifted the spectrum of nuclease activities of Artemis. With DNA-PK_{cs} present, Artemis efficiently opened about 40% of the hairpins during the time interval (FIG. 5A, lanes 4, 5 and 6), however this is probably an underestimation of the hairpin opening activity of Artemis, because once the 5′ radiolabel of the substrate is cleaved, the hairpin opening product becomes invisible on the gel. This result strongly suggests that DNA-PK_{cs} regulates Artemis activity to include hairpin opening, as well as endonucleolytic cleavage of overhangs.

The position of the hairpin opening varied, but a 3′ overhang was preferentially generated at the opened end. As shown in FIG. 5A (lanes 4-8), the predominant hairpin opening was at the +2 position, which corresponds to the 23-nucleotide cleavage product (the phosphodiester bond at the hairpin tip is designated 0, with phosphodiester bonds 3′ to the tip numbered +1, +2, etc., and phosphodiester bonds 5′ to the tip numbered −1, −2, etc.). The DNA-PK_{cs} chemical inhibitor, LY294002, reduced the stimulation (FIG. 5A, lanes 7, 8), while the dimethyl sulfoxide solvent (DMSO) in which LY294002 was dissolved in had little effect (FIG. 5A, lane 6).

To further confirm the importance of DNA-PK_{cs} kinase activity for the hairpin opening and endonucleolytic activities of Artemis:DNA-PK_{cs} complex, non-hydrolyzable ATP analogs were tested in an Artemis nuclease assay. The results are shown in FIG. 5B. Lane M in (A) and (B) contains an oligonucleotide identical to the fragment 5′ to the hairpin tip (21 nucleotides). Sizes of the major products are indicated on the right. Diagrams adjacent to the sizes reflect the hairpin opening positions relative to the substrate. As described above, neither DNA-PK_{cs} nor Artemis alone showed any hairpin opening activity (FIG. 5B, lanes 2 and 3). In the presence of ATP, DNA-PK_{cs} was able to confer Artemis efficient hairpin opening activity (FIG. 5B, lane 4). However, this effect of DNA-PK_{cs} was largely suppressed when ATP-γ-S (FIG. 5B, lane 5) or AMP-PNP (FIG. 5B, lane 6) was used instead of ATP. This indicates the DNA-PK_{cs} kinase activity is critical for the Artemis:DNA-PK_{cs} complex, consistent with the result that Artemis is the substrate of DNA-PK_{cs} in vitro as discussed above with respect to FIG. 10.

While not wishing to be bound by any particular theory, it is believed that the nucleolytic properties of the Artemis:DNA-PK_{cs} complex reside within the complex of these two proteins rather than any other protein co-purifying with one of them for several reasons. First, the DNA-PK_{cs} preparation is devoid of any nuclease activity (FIG. 4B, lane 8, and FIG. 5B, lane 2) (West et al., 1998; Yaneva et al., 1997). Therefore, the hairpin opening activity, the overhang nucleolytic activity, and the 5′ to 3′ exonuclease activities of Artemis are not the result of a contaminating nuclease from the DNA-PK_{cs} preparation. Second, a SCID patient with a single homozygous point mutation of Artemis in the conserved SNM1 domain has been identified, and the phenotype of this patient is indistinguishable from those with null mutations of Artemis (U. Pannicke and K. Schwarz. unpublished). Third, a D165N point mutant of Artemis lacks any hairpin opening activity (FIG. 6). This was cloned into the same expression vector and purified along with the wild-type Artemis protein. The results are shown in FIG. 6, where lanes 3 and 4 show the activity of the D165N mutant and lanes 5 and 6 show the activity the wild type Artemis. Lane M contains a marker oligonucleotide identical to the fragment 5′ to the hairpin tip. Sizes of the major products are indicated on the right.

While still having the 5′ to 3′ exonuclease activity, the point mutant is completely devoid of hairpin opening activity in the presence of DNA-PK_{cs} (FIG. 6, lane 4), similar to the GST-tagged Artemis (see below). These observations strongly support the view that the nucleolytic properties described here reside within the Artemis moiety of the Artemis:DNA-PK_{cs} complex.

### EXAMPLE 14

### DNA-PK_{cs} Regulation of Artemis is ATP-Dependent and Requires the Physical Presence of DNA-PK_{cs}

Tests were performed to determine whether phosphorylation of Artemis by DNA-PK_{cs} is necessary to confer hairpin opening activity on Artemis. A 20 bp artificial hairpin with a 6 nucleotide 5′ overhang end-labeled with T4 PNK and an entirely GC-hairpin was used as the substrate. The results are shown in FIG. 7. Reactions without pre-phosphorylation (lanes 2 to 6) were carried out such that the indicated reagents were mixed with the substrate at the same time. In reactions with pre-phosphorylation (lanes 7 to 11), the indicated reagents were mixed with Artemis-myc-His immunobeads first and incubated to allow the phosphorylation of Artemis; then DNA-PK_{cs} and other reagents were washed away from the immunobeads. The nuclease assay was performed with the treated beads (but without DNA-PK_{cs}, etc.) and the substrate. The “(+)” symbols in the chart above lanes 7 through 11 indicate that these reagents were present only in the pre-phosphorylation of Artemis and not in the nuclease reactions. Sizes of the major products are indicated on the right. Diagrams adjacent to the sizes reflect the hairpin opening positions relative to the substrate.

As shown in FIG. 7, Artemis alone cleaved the hairpin only at the 5′ overhang (non-hairpin end) (lane 2). The Artemis:DNA-PK_{cs} complex opened the hairpin 3′ to the tip at the +1 and +2 positions (lane 5), similar to the positions of hairpin opening of the D_{FL16.1} hairpin as described above. The hairpin opening in lane 5 of FIG. 7, while present is clearly less abundant than that seen for the hairpin shown as shown in FIG. 5A, lane 4, even though the 5′ exonuclease and overhang endonuclease action is equally strong. This may be due to the inefficiency of cleavage of the GC-hairpin end. Hence, the sequence of the hairpin may affect the efficiency of hairpin opening.

The hairpin opening and the overhang cleavage were increased in the presence of ATP (FIG. 7, lane 5) relative to the level when ADP was present (FIG. 7, lane 3). A lower level of endonucleolytic activity both on hairpins and 5′ ends was observed in the presence of ADP, but this may be attributable to low levels of contaminating ATP which are present in ADP. Therefore, ATP is important for the regulation of Artemis by DNA-PK_{cs}, consistent with the finding above that ATP-γ-S and AMP-PNP are unable to replace ATP in the hairpin opening assay of the Artemis:DNA-PK_{cs} complex, as shown in FIG. 5B.

The hairpin opening and the overhang endonucleolytic cleavage were both stimulated by addition of 35 bp dsDNA (FIG. 7, lanes 4 versus lane 5). This was consistent with the dsDNA end stimulation of kinase activity of DNA-PK_{cs}. The equilibrium binding affinity of DNA-PK_{cs} for dsDNA is approximately 10⁻⁹ M (West et al., 1998), and the additional dsDNA permits a higher occupancy and, hence, stimulation of DNA-PK_{cs} Interestingly, although Ku increases the affinity of DNA-PK_{cs} to a DNA end, the presence of Ku did not affect the ability of DNA-PK_{cs} to regulate Artemis (FIG. 7, lane 6). Neither Ku nor DNA-PK_{cs} showed any nuclease activity on this (or other) substrate (data not shown). Based on these studies, it was determined that Artemis phosphorylation by DNA-PK_{cs} is necessary.

Next, to test the possibility that Artemis requires not only phosphorylation by DNA-PK_{cs} but also continued physical complex formation with DNA-PK₅, bead-immobilized Artemis was treated with DNA-PK_{cs} first, and then the extensively washed beads (presumably containing immobilized and phosphorylated Artemis, devoid of any DNA-PK_{cs}) were used for the nuclease assay. It was found that Artemis nuclease activity was equivalent to that of unphosphorylated Artemis, including failure to endonucleolytically open hairpins (FIG. 7, lanes 8 to 11). Hence, the hairpin opening activity is dependent not only on the phosphorylation but also the physical presence of DNA-PK_{cs}. That is, the hairpin opening activity is strictly a property of the Artemis:DNA-PK_{cs} complex, not of Artemis alone and not of DNA-PK_{cs} alone.

The action of Artemis at the 6-nucleotide 5′ overhang of the hairpin was also altered by the presence and kinase activity of DNA-PK_{cs}. Artemis alone removed only 5′ mononucleotides (FIG. 7, lane 2). However, the Artemis:DNA-PK_{cs} complex preferentially removed 5 and 6 nucleotide products in the presence of ATP (FIG. 7, lanes 5, 6). This is consistent with the overhang endonucleolytic cleavage activity described above (FIG. 4A). Given the length of this 5′ overhang, the 5 and 6 nucleotide cleavage products were expected. Furthermore, the pre-phosphorylated Artemis only generated mononucleotide products instead of the 5 and 6 nucleotide overhang cleavage products (FIG. 7, lanes 10 and 11), indicating that the presence of DNA-PK_{cs} is also important for stimulating the overhang endonuclease activity of Artemis.

The GST-Artemis has identical 5′ to 3′ exonucleolytic and overhang endonuclease properties to Artemis-myc-His, except that GST-Artemis is distinctly weaker in activity (about 10-fold), and GST-Artemis fails to open hairpins at any detectable level, even though it is able to form a complex with DNA-PK_{cs} (FIG. 2A). This raises the possibility that the N-terminal region of Artemis is important, perhaps for conformational reasons, for all of the nucleolytic activities of Artemis. This is consistent with the fact that the SNM1 homologous region of Artemis resides in the N-terminal region (Moshous et al., 2001).

### EXAMPLE 15

### The Artemis:DNA-PK_{cs} Complex can Open Rag-Complex Generated Hairpins

In order to test the hypothesis that the Artemis:DNA-PK_{cs} complex could open hairpins generated by the RAG complex (RAG-1, RAG-2 and HMG1), a hairpin-opening experiment was carried out with three different configurations as shown in FIG. 8, where the reaction schemes are shown as brief flow charts. The diagrams for the substrate and the hairpin are shown as base-paired to emphasize the native structures.

In one configuration, the DNA was phenol/chloroform extracted after RAG complex treatment, before exposure of any RAG-generated hairpins to the Artemis:DNA-PK_{cs} complex. In a second configuration, no organic extraction was included, but the Artemis:DNA-PK_{cs} complex was added after the RAG complex had generated hairpins. In the third configuration, the RAG complex and the Artemis:DNA-PK_{cs} complex were added simultaneously. The starting DNA substrate for all three configurations was a radiolabeled 12-RSS substrate accompanied by an unlabeled 23-RSS substrate; this permits the reaction to proceed according to the 12/23 rule, which is essential for efficient hairpin formation in V(D)J recombination in vivo and in vitro. In reactions with multiple steps, substrates were incubated with the RAG complex first, followed by phenol/chloroform extraction (lanes 1 to 4) or no organic extraction (lanes 5 to 8), and then Artemis-myc-His and DNA-PK_{cs} were added. In the one-step reactions (lanes 9 to 12), all proteins were added to the reaction at the start of the incubation. Synthetic oligonucleotides identical to the RAG-generated hairpin and the hairpin tip opening product were co-electrophoresed in lane M. Sizes of the major products are indicated on the right. The diagrams for the substrate and the hairpin are shown as base-paired to emphasize the native structures. The top (unlabeled) strand of the substrate and the fragment of the hairpin that originates from the top strand are depicted as open bars.

As shown in FIG. 8, hairpin formation by the RAG complex was efficient in all of the reactions (lanes 2 to 4, 6 to 8 and 10 to 12). Hairpin opening was also detectable for all three experimental configurations (lanes 4, 8 and 12), indicating that the RAG post-cleavage complex does not block the Artemis:DNA-PK_{cs} complex from opening the hairpins efficiently. The size of the hairpin opening products (FIG. 8, lanes 4, 8 and 12) is consistent with the fact that Artemis opens hairpins 3′ to the tip (FIGS. 5A, 5B, 6 and 7). This is the first efficient opening of RAG-generated hairpins by any vertebrate nuclease in magnesium ion-containing solutions.

### EXAMPLE 16

### Endonucleolytic Structure Specificities of Artemis:DNA-PK_{cs} for DNA Hairpin Opening and for Single-Stranded Overhangs

The positional preferences for the 5′ overhang, 3′ overhang, and hairpin endonucleolytic activities of Artemis:DNA-PK_{cs} are illustrated in FIG. 9. These preferences may have a unifying explanation. With 5′ overhangs (FIG. 9A), the endonucleolytic cleavage preference is directly at the single-strand/double-strand transition point (see also FIG. 4A). With 3′ overhangs (FIG. 9B), the endonucleolytic cleavage preference is displaced −4 nucleotides into the single-stranded region (see also FIG. 4B). Hence, it appears that Artemis:DNA-PK_{cs} recognizes 4 nucleotides of ssDNA (nearest to a double-strand transition) in an orientation-dependent manner (FIG. 9A, B, thick arrows), and it preferentially cleaves at the 3′ side of that 4 nucleotide ssDNA region.

Nuclear magnetic resonance data suggests that DNA hairpins have unpaired bases near the tip, resulting in a 2 to 4 nucleotide single-stranded loop at the tip (FIG. 9C) (Blommers et al., 1989; Howard et al., 1991; Raghunathan et al., 1991). As discussed above with respect to the results shown in FIGS. 5A, 5B, 6, 7, and 8, in the study of the hairpin substrates the endonucleolytic preference is −2 nucleotides 3′ to the hairpin tip. If one considers the hairpin as a single-stranded 5′ extension of the bottom strand (FIG. 9D), then the overhang studies (FIGS. 4A and 7) would predict preferential cleavage 3′ of the fourth nucleotide in a 4 nucleotide hairpin loop. This is the region where the hairpin opening preference was observed in the studies presented herein. Likewise, if one regards the hairpin as a 3′ extension of the top strand (FIG. 9E), then one would predict cleavage 3′ of the four single-stranded nucleotides at the hairpin end, based on the overhang studies (FIG. 4B). This, again, is the same preferred position as observed in the hairpin opening studies discussed above. Therefore, the 5′ and 3′ overhang studies both predict the same positional preference in the hairpin, and that is where the observed preferential cleavage occurs (FIGS. 9C-E). This suggests that the Artemis moiety of the Artemis:DNA-PK_{cs} complex recognizes an approximately 4 nucleotide single-stranded region of the hairpin tip and cleaves 3′ to that 4 nucleotide region (FIG. 9C). The obvious variation in cleavage around these preferential sites was noted. Moreover, the binding of Ku and other proteins may result in greater lengths of hairpin melting, and this might permit Artemis:DNA-PK_{cs} to cleave more internally, thereby causing deletions deeper into the coding end. The variation in coding end nucleotide loss is known to have clear evolutionary utility for V(D)J recombination.

The invention may be embodied in other specific forms without departing from its essential characteristics. The described embodiments are to be considered in all respects only as illustrative and not as restrictive. Indeed, those skilled in the art can readily envision and produce further embodiments, based on the teachings herein, without undue experimentation. The scope of the invention is, therefore, indicated by the appended claims rather than by the foregoing description. All changes that come within the meaning and range of the equivalence of the claims are to be embraced within their scope.

### REFERENCES

Agrawal, A., and Schatz, D. G. (1997). RAG1 and RAG2 form a stable postcleavage synaptic complex with DNA containing signal ends in V(D)J recombination. Cell, 89:4353.
Anderson, C. W., and Carter, T. H. (1996). The DNA-Activated Protein Kinase-DNA-PK. In Molecular Analysis of DNA Rearrangements in the Immune System, R. Jessberger and M. R. Lieber, eds. (Heidelberg: Springer-Verlag), pp. 91-112.
Barnes, D. E., Stamp, G., Rosewell, U, Denzel, A., and Lindahl, T. (1998). Targeted disruption of the gene encoding DNA ligase IV leads to lethality in embryonic tissues. Curr. Biol., 8: 1395-1398.
Besmer, E., Mansilla-Soto, J., Cassard, S.; Sawehuk, D. J., Brown, G., Sadofsky, M., Lewis, S. M., Nussenzweig, M. C., and Comes, P. (1998). Hairpin coding end opening is mediated by the recombination activating genes RAG1 and RAG2. Molecular Cell, 2: 817-828.
Blommers, M. J., Walters, J. A., Haasnoot, C. A., Aelen, J. M., Marel, G. v., Boom, J. v., and Hilbers, C. W. (1989). Effects of base sequence on the loop folding in DNA hairpins. Biochemistry, 28: 7491-7498.
Blunt, T., Finnie, N. J., Taccioli, G. E., Smith, G. C. M., Demengeot, J., Gottlieb, T. M., Mizuta, R., Varghese, A. J., Alt, F. W., Jeggo, P. A., and Jackson, S. P. (1995). Defective DNA-Dependent Protein Kinase Activity is Linked to V(D)J Recombination and DNA Repair Defects Associated with the Murine SCID Mutation. Cell, 80: 813-823.
Bosma, M. J., and Carroll, A. M. (1991). The scid mouse mutant: Definition, characterization, and potential uses. Ann. Rev. Immun., 9: 323-350.
Chan, D. W., Mody, C. H., Ting, N. S., and Lees-Miller, S. P. (1996). Purification and characterization of the double-stranded DNA-activated protein kinase, DNA-PK, from human placenta. Biochem. Cell Biol., 74: 67-73.
Chiu, C. Y., Cary, R. B., Chen, D. J., Peterson, S. R., and Steward, P. L. (1998). CryoEM imaging of the catalytic subunit of the DNA-dependent protein kinase. J. Mol. Biol., 284: 1075-1081.
Comes, P., Weis-Garcia, F., Misulovin, Z., Nussenzweig, A., Lai, J., Li, G., Nussenzweig, M., and Baltimore, D. (1996). In vitro V(D)J recombination: signal joint formation. Proc. Natl. Acad. Sci., 93: 14008-14013.
Dronkert, M. L. G., Wit, J. d., Boeve, M., Vasconcelos, M. L., Steeg, H. v., Tan, T. L., Hoeijmakers, J. H. J.; and Kanaar, R. (2000). Disruption of mouse SNM1 causes increased sensitivity to the DNA interstrand cross-linking agent mitomycin C. Mol. Cell. Biol., 20: 4553-4561.
Fugmann, S. D., Lee, A. L, Shockett, P. E., Villey, I. J., and Schatz, D. G. (2000). The RAG proteins and V(D)J recombination: complexes, ends, and transposition. Ann. Rev. Immunol., 18: 495-527.
Gao, Y., Sun, Y., Frank, K., Dikkes, P., Fujiwara, Y., Seidl, K., Sekiguchi, J., Rathbun, G., Swat, W., Wang, J., Bronson, R., Malynr˜ B., Bryans, M., Zhu, C., Chaudhuri, J., Davidson, L., Ferrini, R., Stamato, T., Orkin, S. H., Greenberg, M. E., and Alt, F. W. (1998). A critical role for DNA end joining proteins in both lymphogenesis and neurogenesis. Cell, 95: 891-902.
Gauss, G. H., and Lieber, M. R. (1996). Mechanistic constraints on diversity in human V(D)J recombination. Mol. Cell. Biology, 16: 258-69.
Gellert, M. (1997). Recent advances in understanding V(D)J recombination. Adv. Immunol., 64: 39-64.
Grawunder, U., West, R. B., and Lieber, M. R. (1998). Antigen Receptor Gene Rearrangement. Curr. Op. Immunol., 10: 172-180.
Grawunder, U., Wilin, M., Wu, X., Kulesza, P., Wilson, T. E., Mann, M., and Lieber, M. R. (1997). Activity of DNA ligase IV stimulated by complex formation with XRCC4; protein in mammalian cells. Nature, 388: 492-495.
Grawunder, U., Zimmer, D., Fugmann, S., Schwarz, K., and Lieber, M. R. (1998). DNA ligase IV is essential for V(D)J recombination and DNA double-strand break repair in human precursor lymphocytes. Mol. Cell, 2: 477-484.
Hammarsten, O., and Chu, G. (1998). DNA-Dependent Protein Kinase: DNA Binding and Activation in the Absence of Ku. Proc. Natl. Acad. Sci., 95: 525-530.
Hanakahi, L., Bartlet-Jones, M., Chappell, C., Pappin, D., and West, S. C. (2000). Binding of inositol phosphate to DNA-PK and stimulation of double-strand break repair. Cell, 102: 721-729.
Harfst, E., Cooper, S., Neubauer, S., Distel, L., and Grawunder, U. (2000). Normal V(D)J recombination in cells from patients with Nijmegen breakage syndrome. Mol. Immunology, 37: 915-929.
Harrington, J., Hsieh, C.-L., Gerton, J., Bosma, G., and Lieber, M. R. (1992). Analysis of the defect in DNA end joining in the marine scid mutation. Mol. Cell. Biol., 12: 47584768.
Hendrickson, E. A., Qin, S.-Q., Bump, E. A., Schatz, D. G., Oettinger, M., and Weaver, D. T. (1991): A link between double-strand break-related repair and V(D)J recombination: The scid mutation. Proc. Natl. Acad. Sci., 88: 4061-4065.
Henriques, J. A.; and Moustacchi, E. (1980). Isolation and characterization of pso mutants sensitive to photo-addition of psoralen derivatives in S. cerevisiae. Genetics, 95: 273-288.
Hiom, K., and Gellert, M. (1997). A stable RAG 1-RAG 2-DNA complex that is active in V(D)J cleavage. Cell, 88: 65-72.
Howard, F. B., Chen, C., Ross, P. D., and Miles, H. T. (1991). Hairpin formation in the self-complementary dodecamer d-GGTACGCGTACC and derivatives containing GA and IA mispairs. Biochemistry, 30: 779-782.
Kowalczykowski, S. C., and Eggleston, A. K. (1994). Homologous pairing and DNA strand-exchange proteins. Ann. Rev. Biochem., 63: 991-1043.
Leather, K. K., Hammarsten, O., Kornberg, R. D., and Chu, G. (1999). Structure of the DNA-dependent protein kinase: implications for its regulation by DNA. EMBO J., 18:, 1114-1123.
Lewis, S. M. (1994). The Mechanism of V(D)J Joining: Lessons from Molecular, Immunological and Comparative Analyses. Adv. Imm., B: 27-150.
Lieber, M. R. (1999). The biochemistry and biological significance of nonhomologous DNA end joining: an essential repair process in multicellular eukaryotes. Genes Cells, B: 77-85.
Lieber, M. R. (1997). The FEN-1 family of structure-specific nucleases in eukaryotic DNA replication, recombination, and repair. BioEssays, 19:233-240.
Lieber, M. R. (1998). Pathologic and Physiologic Double-Strand Breaks: Roles in Cancer, Aging, and the Immune System. Am. J. Path., 153: 1323-1332.
Lieber, M. R. (1991). Site-specific recombination in the immune system. FASEB J., 5, 2934-2944.
Lieber, M. R., Hesse, J. E., Lewis, S., Bosma, G. C., Rosenberg, N., Mizuuchi, K., Bosma, M. J., and Gellert, M. (1988). The defect in marine severe combined immune deficiency: joining of signal sequences but not coding segments in V(D)J recombination. Cell, 55: 7-16.
Lin, J. M., Landree, M. A., and Roth, D. B. (1999). V(D)J recombination catalyzed by mutant RAG proteins lacking consensus DNA-PK phosphorylation sites. Mol. Immunol, 36: 1263-1299.
Lobachev, K. S., Gordenin, D. A., and Resnick, M. A. (2002). The Mre11 complex is required for repair of hairpin-capped double-strand breaks and prevention of chromosome rearrangements. Cell, 108: 183-193.
Ma, Y., and Lieber, M. R. (2002). Binding of inositol hexakisphosphate (IP6) to Ku but Not to DNA-PK_{cs}. J. Biol. Chem., 277 (in press).
Ma, Y., and Lieber, M. R. (2001). DNA length-dependent cooperative interaction in the binding of Ku to DNA. Cell, 40: 9638-9646.
McCormack, W., Tjoelker, L., Carlson, L., Petryniak, B., Barth, C., Humphries, E. and Thompson, C. (1989). Chicken IgL gene rearrangement involves deletion of a circular episome and addition of single nonrandom nucleotides to both coding segments. Cell, 56: 785-791.
Modesti, M., Hesse, Jo., and Gellert, M. (1999). DNA binding of XRCC4 is associated with V(D)J recombination but not with stimulation of DNA ligase TV activity. EMBO J, 18: 2008-2018.
Moshous, D., Callebaut, L, Chasseval, R. d., Corneo, B., Cavazzana-Calvo, M., Diest, F. L., Tezcan, L, Sanal, O., Bertrand, Y., Philippe, N., Fischer, A., and Villartay, J.-P. d. (2001). Artemis, a novel DNA double-strand break repair/V(D)J recombination protein, is mutated in human severe combined immune deficiency. Cell, 105: 177-186.
Moshous, D., Li, L., Chasseval, R., Philippe, N., Jabado, N., Cowan, M. J., Fischer, A., and Villartay, J.-P. d. (2000). A new gene involved in DNA double-strand break repair and V(D)J recombination is located on human chromosome 10p. Hum. Mol. Gen., 9: 583588.
NickMcElhinny, S. A., Snowden, C. M., McCarville J., and Ramsden, D. A. (2000). Ku Recruits the XRCC4-Ligase IV Complex to DNA Ends. Mol. Cell. Biol., 20: 2996-3003.
Nicolas, N., Moshous, D., Cavazzana-Calvo, M., Papadoupoulo, D., Chasseval, R. d., Deist, F. L., Fischer, A., and Villartay, J.-P. d. (1998). A human severe combined immunodeficiency condition with increased sensitivity to ionizing radiation and impaired V(D)J rearrangements defines a new DNA recombination/repair deficiency. J. Exp. Med., 188: 627-634.
Paul, W. (1999). Fundamental Immunology, 4th Edition (New York: Raven Press).
Paull, T., and Gellert, M. (1998). The 3′ to 51 exonuclease activity of Mre11 facilitates repair of DNA double-strand breaks. Mol. Cell, 1:969-979.
Paull, T. T., and Gellert, M. (1999). Nbs1 potentiates ATP-driven DNA unwinding and endonuclease cleavage by the Mre11/Rad50 complex. Genes Dev., 13: 1276-1288.
Raghunathan, G., Jernigan, R. L., Miles, H. T., and Sasisekharan, V. (1991). Conformational feasibility of a hairpin with two purines in the loop. 5′-dGGTACIAGTACC-3′. Biochemistry, 30:782-788.
Roth, D. B., Menetski, J. P., Nakajima, P., Bosma, M. J., and Gellert, M. (1992). V(D)J recombination: broken DNA molecules with covalently sealed (hairpin) coding ends in SCID mouse thymocytes. Cell, 70:983-991.
Sawchuk, D., Weis-Garcia, F., Malik, S., Besmer, E., Bustin, M., Nussenzweig, M., and Cortes, P. (1997). V(D)J recombination: modulation of RAG1 and RAG2 cleavage activity on 12/23 substrates by whole cell extract and DNA-bending proteins. J. Exp. Med., 185: 2025-2032.
Schar, P., Herrmann, G., Daly, G., and Lindahl, T. (1997). A newly identified DNA ligase of S. cerevisiae involved in RAD52-independent repair of DNA double-strand breaks. Gene Devel., 11: 1912-1924.
Schlissel, M. S. (1998). Structure of nonhairpin coding-end DNA breaks in cells undergoing V(D)J recombination. Mol. Cell. Biol., 18: 2029-2037.
Schuler, W., Weiler, I. J., Schuler, A., Phillips, R. A., Rosenberg, N., Mak, T. W., Kearney, J. F., Perry, R. P., and Bosma, M. J. (1986). Rearrangement of antigen receptor genes is defective in mice with severe combined immune deficiency. Cell, 46: 963-972.
Schwarz, K., Gauss, G. II., Ludwig, L., Pannicke, U., Li., Z., Lindner, D., Friedrich, W., Seger, R. A., Hansen, H. T., Desiderio, S., Lieber, M. R., and Bartram, C. R. (1996). RAG mutations in human B cell-negative SCID. Science, 274: 97-9.
Schwarz, K., Hansen-Hagge, T. E., Knobloch, C., Friedrich, W., Kleihauer, E., and Bartran, C. R. (1991). Severe combined immunodeficiency (SCID) in man: B cell-negative (B−) SCID patients exhibit an irregular recombination pattern at the Jg locus. J. Exp. Med., 174: 1039-1048.
Shen, B., Nolan, J. P., Sklar, L. A., and Park, M. S. (1996). Essential amino acids for substrate binding and catalysis of human flap endonuclease 1. J. Biol. Chem., 271: 9173-9176.
Shockett, P. E., and Schatz, D. G. (1999). DNA hairpin opening mediated by the RAG 1 and RAG2 proteins. Mol. Cell. Biol., 19: 4159-4166.
Teo, S. H., and Jackson, S. P. (1997), Identification of S. cerevisiae DNA ligase IV: involvement in DNA double-strand break repair. EMBO J., 16: 4788-4795.
Vanasse, G. J., Concannon, P., and Willerford, D. M. (1999). Regulated genomic instability and neoplasia in the lymphoid lineage. Blood, 94: 3997-4010.
vanGent, D. C., Hoeijmakers, J. H. J., and Kanaar, R. (2001). Chromosomal stability and the DNA double-stranded break connection. Nat. Rev. Genet, 2: 196-206.
vanGent, D. C., Mizuuchi, K., and Gellert, M. (1996). Similarities between initiation of V(D)J recombination and retroviral integration: Science, 271: 1592-1594.
Villa, A., Sobacchi, C., Notarangelo, L., Bozzi, F., Abinun, M., Abrahamsen, t., Arkwright, P., Baniyash, M., Brooks, E., Conley, M., Comes, P., Duse, M., Fasth, A., Filopovich, A., Infante, A., Jones, A., Mazzolari, E., Muller, S., Pasic, S., Rechavi, G., Sacco, M., Santagata, S., Schroeder, M., Seger, R., Strina, D., Ugazio, A., Valiaho, J., Vihinen, M., Vogler, L., Ochs, H., Vezzoni; P., Fredrich, W., and Schwarz, K. (2001). V(D)J recombination defects in lymphocytes due to RAG mutations: severe immunodeficiency with a spectrum of clinical presentations. Blood, 97: 81-88.
Walker, J. R., Corpina, R. A., and Goldberg, J. (2001). Structure of the Ku heterodimer bound to DNA and its implications for double-strand break repair. Nature, 412:607-614.
West, C. E., Waterwomh, W. M., Jiang, Q., and Bray, C. M. (2000). Arabidopsis DNA ligase IV is induced by gamma-irradiation and interacts with an Arabidopsis homologue of the double-strand break repair protein XRCC4. Plant J., 24: 67-78.
West, R. B., and Lieber, M. R. (1998). The RAG-HMG1 Complex Enforces the 12/23 Rule of V(D)J Recombination Specifically at the Double-Hairpin Formation Step. Mol. Cell. Biol., 18: 6408-6415.
West, R. B.; Yaneva, M., and Lieber, M. R. (1998). Productive and Nonproductive Complexes of Ku and DNA-PK at DNA Termini. Mol. Cell. Biol., 18: 5908-5920.
Wigler, M., Sweet, R., Kim, G. K., Wold, B., Pellicer, A., Lacy, E., Maniatis, T., Silverstein, S., and Axel, R. (1979). Transformation of mammalian cells with genes from procaryotes and eucaryotes. Cell, 16:777-785.
Wilson, T., and Lieber, M. R. (1999). Efficient processing of DNA ends during yeast nonhomologous end joining: evidence for a DNA polymerase beta (POL4)-dependent pathway. J. Biol. Chem., 274: 23599-23609.
Wilson, T. E., Grawunder, U., and Lieber, M. R. (1997). Yeast DNA ligase IV mediates non-homologous DNA end joining. Nature, 388: 495-498.
Wood, R. D., Mitchell, M., Sgouros, M., and Lindahl, T. (2001). Human DNA repair genes. Science, 291:1284-1289.
Wu, X., Wilson, T. E., and Lieber, M. R. (1999). A Role for FEN-1 in Nonhomologous DNA End Joining. Proc. Natl. Acad. Sca. USA, 1303-1308.
Xie, Y., Liu, Y., Argueso, J. L., Hendriksen, L. A., Kao, H., Bambara, R. A., and Alani, E. (2001). Identification of rad27 mutations that confer differential defects in mutation avoidance, repeat tract instability, and flap cleavage. Mol. Cell. Biol, 21: 4889-4899.
Yaneva, M., Kowalewski, T., and Lieber, M. R. (1997). Interaction of DNA-dependent protein kinase with DNA and with Ku: biochemical and atomic-force microscopy. EMBO J., 16:5098-5112.
Yeo, T. C., Xia, D., Hassouneh, S., Yang, X. O., Sabath, D. E., Sperling, K., Gatti, R. A., Concannon, P., and Willerford, D. M. (2000). V(D)J rearrangement in Nijmegen breakage syndrome. Mol. Immunol., 37:1131-1139.
Yu, K., and Lieber, M. R. (2000). The Nicking Step of V(D)J Recombination is Independent of Synapsis: Implications for the Immune Repertoire. Mol. Cell. Biol., 20: 7914-7921.
Zhu, C., Bogue, M. A., Lim, D.-S., Hasty, P., and Roth, D. B. (1996). Ku86-deficient mice exhibit severe combined immunodeficiency and defective processing of V(D)J recombination intermediates. Cell, 86: 379-389.

1. An exonucleolytic composition, consisting essentially of Artemis.

2. An exonucleolytic composition, consisting essentially of Artemis and magnesium ions.

3. A method of exonucleolytically cleaving a single-stranded nucleotide, comprising contacting said nucleotide with a composition consisting essentially of Artemis or a composition consisting essentially of Artemis and magnesium ions under conditions that allow Artemis to cleave said nucleotide.

4. The method of claim 3, wherein said single-stranded nucleotide is a 5′ overhang of a double-stranded DNA.

5. The method of claim 3, wherein said single-stranded nucleotide is a mismatched sequence of a branched double-stranded DNA.

6. The method of claim 3, wherein said single-stranded nucleotide is RNA or DNA.

7. The method of claim 3, wherein said nucleotide comprises a 5′ phosphate.

8. (canceled)

9. (canceled)

10. (canceled)

11. (canceled)

12. A method of endonucleolytically cleaving a nucleotide having hairpin motif comprising a single-stranded loop, said method comprising contacting said nucleotide with a composition comprising a Artemis:DNA-PK_{cs} complex under conditions that allow said Artemis:DNA-PK_{cs} complex to cleave said nucleotide, wherein said cleavage occurs at the beginning of said or at a position within said loop.

13. The method of claim 12, wherein cleavage occurs at a position from about 1-4 nucleotides 5′ from the start of said loop.

14. The method of claim 12 wherein cleavage occurs at a position from about 1-4 nucleotides 3′ from the start of said loop.

15. The method of claim 12, wherein the hairpin is generated by a RAG complex comprising a 12-nucleotide recombination signal sequence/23-nucleotide recombination signal sequence substrate pair.

16. The method of claim 12, wherein said conditions include adding a phosphorylating agent.

17. The method of claim 16, wherein said phosphorylating agent is ATP.

18. The method of claim 12, wherein said conditions include adding a buffer containing magnesium ions.

19. A method of endonucleolytically cleaving a 5′ or 3′ single-stranded nucleotide overhang on a double-stranded DNA, comprising combining said nucleotide with a composition comprising an Artemis:DNA-PK_{cs} complex under conditions that allow said Artemis:DNA-PK_{cs} complex to cleave said overhang.

20. The method of claim 19, wherein said cleavage occurs at the junction between the single-stranded overhang and the double-stranded DNA.

21. The method of claim 19, wherein said cleavage occurs at a position 1 to 10 nucleotides from the junction between the single-stranded overhang and the double-stranded DNA.

22. The method of claim 19, wherein said conditions include adding a phosphorylating agent.

23. The method of claim 19, wherein said conditions include adding magnesium ions.

24. A method of analyzing a nucleic acid suspected of containing a hairpin motif said method comprising:
(a) providing a composition comprising an Artemis:DNA-PK_{cs} complex;
(b) contacting said complex with said nucleic acid under conditions that allow said complex to cleave and open hairpin motifs; and
(c) analyzing said nucleic acid by gel electrophoresis, fluorescence-based methods or radioactivity-based methods.

25. The method of claim 24, wherein said conditions include adding a phosphorylating agent.

26. The method of claim 24, wherein said conditions include adding magnesium ions.

27. A method of producing a fusion protein containing Artemis, said method comprising:
(a) providing an expression vector comprising a nucleic acid sequence that encodes an affinity tag;
(b) inserting a polynucleotide that encodes Artemis into said vector in a manner that allows said polynucleotide to be operatively linked to said vector; and
(c) transfecting cells with said vector under conditions that allow expression of said Artemis and said affinity tag to produce said fusion protein comprising Artemis linked to said affinity tag.

28. The method of claim 27, further comprising:
(d) contacting said fusion protein with a matrix comprising a compound that binds said affinity tag under conditions that allow said compound to bind said affinity tag; and
(e) recovering said fusion protein to provide a purified fusion protein.

29. The method of claim 28, wherein said affinity tag is glutathione-S-transferase and said matrix is GSH-agarose.

30. The method of claim 28, wherein said affinity tag is myc-his, and said matrix is Ni-nitrilotriacetic acid agarose.

31. The method of claim 27 wherein said fusion protein further comprises DNA-PK_{cs} linked to said Artemis, said method further comprising inserting a gene that encodes DNA-PK_{cs} into said vector at a position adjacent said gene that encodes Artemis.

32. A method for screening a compound effective as an inhibitor of Artemis, the method comprising:
(a) preparing a reaction mixture by combining Artemis with or without DNA-PK_{cs} and with at least one test compound under conditions permissive for the activity of Artemis for a predetermined length of time;
(b) assessing the activity of Artemis with or without DNA-PK_{cs} and in the presence of the test compound after said predetermined length of time; and
(c) comparing the activity of Artemis with or without DNA-PK_{cs} and in the presence of the test compound with the activity of Artemis with or without DNA-PK_{cs} and in the absence of the test compound, wherein a decrease in the activity of Artemis in the presence of the test compound is indicative of a compound that acts as an inhibitor of Artemis.

33. The method of claim 32, wherein said activity is measured after said predetermined length of time by contacting said reaction mixture with a double-stranded DNA comprising a terminal single-stranded nucleotide, and determining whether said Artemis exonucleolytically cleaves said single-stranded nucleotide.

34. The method of claim 32, wherein said compound is a compound known to inhibit the activity of beta-lactamase.

35. (canceled)

36. (canceled)

37. (canceled)

38. A method of analyzing a nucleic acid target having a first nucleotide sequence, said method comprising:
(a) providing a nuclease composition having a 5′ to 3′ nuclease activity consisting essentially of Artemis;
(b) contacting the nucleic acid target with said nuclease composition under conditions sufficient to permit the 5′ to 3′ nuclease activity of the polymerase to cleave the nucleotide bonds of the first nucleotide sequence when (1) the first nucleotide sequence is a 3′ or 5′ single stranded overhang or (2) mismatched regions of the first nucleotide sequence when the first nucleotide sequence is in duplex nucleic acid; and
(c) analyzing said nucleic acid target by gel electrophoresis, fluorescent-based methods or radioactivity-based methods.

39. A method of ameliorating a condition caused by the activity of Artemis in a patient, comprising administering to said patient an Artemis inhibitor in an amount effective to inhibit Artemis.

40. The method of claim 39, wherein said condition is cancer.

41. The method of claim 39, wherein said condition is acute lymphoblastic leukemia.

42. A method of enhancing cancer therapy, comprising delivering an Artemis inhibitor to cancerous cells in said patient in an amount effective to inhibit Artemis, followed by administration of a traditional cancer therapy to said patient.

43. A method of diagnosing a disease or condition in a patient associated with an altered or abnormal amount of Artemis, said method comprising:
providing a fluid or tissue sample from said patient; and
measuring the level of Artemis in said sample.

44. The method of claim 43, wherein said level is measured by contacting said sample with a labeled antibody that specifically binds Artemis, wherein said antibody is bound to a substrate and detecting the amount of Artemis that binds to said antibody.
